SEQUENCE LISTING (35 sequences)

SEQ ID NO: 1 - DNA, 2080 bp, Corynebacterium glutamicum
  CDS (209)..(1960)
aagtcactgcccacaagtgactgaacctggcagcgacctcatgaattgtttgaaaaacattttttttgggcatgaaaaggggatacagttagctgcataccggcctttttgggttggcatcggatcctgcctgtggcctaagatcaggcagtgttgttaaaggacgatcggtaatccgaatggatcgtcccgtagtcaggaggaacctatgaccgctgtggcgcctagggtcgacgggcacgtcgcccctcagaggcccgagccgacaggccatgcacgcaagggcagcaaagcatggttaatgatgaccaccaccgaccacaagcagctgggcattatgtacatcattatgtccttcagcttcttcttcctcggtggcttgatggccctgcttatccgagcggagcttttcacccctggtctgcagttcctgtctaatgagcagttcaaccagctgttcaccatgcacggaactgtcatgctgctgctgtacggaactccaattgtttggggttttgctaactacgtcctgccacttcagatcggtgcgcctgacgtagctttcccacgtttgaatgctttcggcttctggatcaccaccgtcggtggtgtcgcgatgctgaccggcttcctgaccccgggtggtgctgccgacttcggttggaccatgtactccccactgtctgacgcaattcactccccaggccttggctctgacatgtggattgtcggtgtcggtgcaactggtattggctccgttgcttccgcaattaacatgctcaccaccatcctctgcctccgcgcacctggtatgaccatgttccgtatgcctattttcacctggaatatcttcgttgtttccgttcttgctctgctgatcttcccactgctgctcgctgctgcactgggtgttctgtatgaccgcaagcttggtggacacctgtacgatccagctaacggcggctccctcctgtggcagcacctgttctggttcttcggacaccctgaggtttacgttctggcgctgccgttcttcggcattgtttctgagatcattcctgtgttctcccgtaagccaatgttcggttacgtcggcctgatcttcgcaaccttgtccattggtgcactgtccatggctgtgtgggctcaccacatgttcgttactggcgcagttttgcttccgttcttctccttcatgacgttcctgatttcggttcctaccggcgttaagttcttcaactgggttggaaccatgtggaagggtcacatcacttgggaaaccccaatgatctggtctgttggcttcatggctaccttcctcttcggtggtctgaccggcattatgctggcgtccccaccactggacttccacttggctgactcctacttcctgatcgcgcacttccactacaccctcttcggtaccgtggtgttcgcatcgtgtgcaggcgtttacttctggttcccgaagatgactggccgcatgatggacgagcgtcttggcaagatccacttctggttgaccttcgtcggtttccacggaaccttcctcatccagcactgggtgggcaacatgggtatgccacgtcgttacgctgactacctggattctgatggtttcaccatctacaaccagatctccaccgtgttctccttcctgcttggcctgtctgtcattccattcatctggaacgtcttcaagtcctggcgctacggtgagctcgttaccgttgatgatccttggggttacggcaactccctggagtgggcaacctcctgccctcctcctcgccacaacttcgcatccttgcctcgtatccgctccgagcgccctgcgttcgagctgcactacccgcacatgattgaacgcatgcgcgcagaggcacacactggacatcacgatgatattaatgctccagaattgggtaccgccccagcccttgcatctgactccagccgctaaaagcgtctgatttaagtcggtacctgactaaataagcaccagccccagcagagataatctgccggggctggtgcttttcatattccgacttggggcacccctgaatacatctcaccc

SEQ ID NO: 2 - protein, 584 aa, Corynebacterium glutamicum
MTAVAPRVDGHVAPQRPEPTGHARKGSKAWLMMTTTDHKQLGIMYIIMSFSFFFLGGLMALLIRAELFTPGLQFLSNEQFNQLFTMHGTVMLLLYGTPIVWGFANYVLPLQIGAPDVAFPRLNAFGFWITTVGGVAMLTGFLTPGGAADFGWTMYSPLSDAIHSPGLGSDMWIVGVGATGIGSVASAINMLTTILCLRAPGMTMFRMPIFTWNIFVVSVLALLIFPLLLAAALGVLYDRKLGGHLYDPANGGSLLWQHLFWFFGHPEVYVLALPFFGIVSEIIPVFSRKPMFGYVGLIFATLSIGALSMAVWAHHMFVTGAVLLPFFSFMTFLISVPTGVKFFNWVGTMWKGHITWETPMIWSVGFMATFLFGGLTGIMLASPPLDFHLADSYFLIAHFHYTLFGTVVFASCAGVYFWFPKMTGRMMDERLGKIHFWLTFVGFHGTFLIQHWVGNMGMPRRYADYLDSDGFTIYNQISTVFSFLLGLSVIPFIWNVFKSWRYGELVTVDDPWGYGNSLEWATSCPPPRHNFASLPRIRSERPAFELHYPHMIERMRAEAHTGHHDDINAPELGTAPALASDSSR

SEQ ID NO: 3 - DNA, 618 bp, Corynebacterium glutamicum
  CDS (1)..(615)
gtgacgagcgcagttggaaatacaggtatggcagcaccacaacgtgttgcggcactgaaccgtccgaatatggtcagtgtcggcaccattgtgttcctgtctcaggaattaatgttcttcgccgggctattcgcgatgtacttcgtgtcccgtgcgaacggactggcaaatggatcatggggagagcagacagatcacctcaacgtgccctacgcactgttgattacggtcattctggtgtcttcctcagtgacttgccagttcggagtttttgcggctgaaaggggtgacgtttacggcctccgcaagtggttcttggtcacgattatcctcggatcaatcttcgtgatcggacagggctacgagtacatcactctcgtaggtcacggacttacaatccagagcagtgtctacggatcggcattctttattacaaccggtttccacgcactgcacgtgatcgcgggtgttatggccttcgttgtggttcttatgagaatccataagtcgaagttcactccggcacaggcaaccgcagcaatggttgtgtcttattactggcacttcgttgacgtggtctggatcggcctcttcatcactatttacttcattcagtag

SEQ ID NO: 4 - protein, 205 aa, Corynebacterium glutamicum
MTSAVGNTGMAAPQRVAALNRPNMVSVGTIVFLSQELMFFAGLFAMYFVSRANGLANGSWGEQTDHLNVPYALLITVILVSSSVTCQFGVFAAERGDVYGLRKWFLVTIILGSIFVIGQGYEYITLVGHGLTIQSSVYGSAFFITTGFHALHVIAGVMAFVVVLMRIHKSKFTPAQATAAMVVSYYWHFVDVVWIGLFITIYFIQ

SEQ ID NO: 5 - DNA, 852 bp, Corynebacterium glutamicum
  CDS (1)..(849)
atggctaaaccctctgctaagaaggtcaagaatcgccgcaaggtccggcgcaccgtcgcaggtgcattggctctgaccattggactgagcggagcaggaatcctcgcaaccgcgatcactccagatgctcaagttgctaccgctcagcgtgacgatcaggcacttatctccgagggtaaagacctctacgatgtcgcctgcatcacctgccacggcgtaaacctccaaggtgttgaggaccgcggtccttccctcgtaggtgttggcgaaggcgcagtgtacttccaagttcactccggccgtatgccaatactgcgtaacgaggctcaggctgagcgcaaggctcctcgttacaccgaggcacagacccttgcgatcgctgcatatgttgcagctaatggcggtggcccaggactcgtttacaacgaggacggcaccctcgccatggaggagctccgtggcgaaaactacgacggacagattacctccgccgacgtcgctcgcggcggagatctgttccgcctgaactgtgcatcctgccacaacttcactggtcgtggtggcgcactgtcctctggtaagtacgcaccaaacctggatgctgcaaacgagcaggaaatctaccaggctatgcttaccggtcctcagaacatgcctaagttctccgatcgtcagctctccgcagatgagaagaaggacatcatcgccttcatcaagtccaccaaggagaccccatcaccaggtggttactcactcggtagcttgggcccagtggctgagggtctgttcatgtgggtattcggcatcttggtcctcgtggccgccgctatgtggattggatcacgttcatga

SEQ ID NO: 6 - protein, 283 aa, Corynebacterium glutamicum
MAKPSAKKVKNRRKVRRTVAGALALTIGLSGAGILATAITPDAQVATAQRDDQALISEGKDLYDVACITCHGVNLQGVEDRGPSLVGVGEGAVYFQVHSGRMPILRNEAQAERKAPRYTEAQTLAIAAYVAANGGGPGLVYNEDGTLAMEELRGENYDGQITSADVARGGDLFRLNCASCHNFTGRGGALSSGKYAPNLDAANEQEIYQAMLTGPQNMPKFSDRQLSADEKKDIIAFIKSTKETPSPGGYSLGSLGPVAEGLFMWVFGILVLVAAAMWIGSRS

SEQ ID NO: 7 - DNA, 1227 bp, Corynebacterium glutamicum
  CDS (1)..(1224)
atgagtaacaacaacgacaaacagtacacaacccaagaactcaacgcgatgagcaatgaggatcttgcacgacttggtacagagctggacgacgttaccattgcataccgcaaggaacgtttcccaatcgctaatgacccagctgagaagcgcgctgcacgtgcagttactttctggctagtcctcggcatcattggtggacttgggttcctggctacctacattttctggccttgggagtacaaggcacacggagatgaaggtctcctggcgtacaccttgtacaccccaatgctgggtattacttccggtctttgcatcctgtccctgggatttgcagttgtcctttatgtcaagaagttcattccagaggaaatcgcagtacagcgtcgccacgacggtccttctgaagaagttgaccgccgcaccatcgttgcacttctcaatgactcttggcagacctctactcttggtcgtcgcaagctgatcatgggacttgcaggtggcggagcagtactggccggcctgaccatcatcgctccaatgggcggtatgatcaagaacccttggaatcctaaggaaggcccaatggacgttcagggtgacggcaccctgtggacttccggttggactctcgttgagaacgacgtcaaggtttacctcggccgcgacactgcagcaattgcggagtcccacaccgatgcaaccggtgagcactggtcaaccactggtgtttcccgcctggttcgtatgcgcccagaagatctggcagcagcatccatggaaactgtcttcccacttccagctgaaatggtgaacgacggtgctgaatacgatcctgcgaaggacgtctacgagcaccaaatgcactcggtgcacggcccacgcaacgcagttatgttgatccgtctccgtaccgctgacgctgaaaaggttatcgaacgcgaaggccaggagtccttccactacggtgactactacgcttactccaagatttgtacacacattggttgcccaacctcactgtacgaggctcagaccaatcgtattctgtgcccatgtcaccagtcgcagtttgacgcattgcactacggaaagccagtctttggacctgctgcccgtgcactgccacagctgccaattaccgttgatgaagagggctacctcatcgccgctggtaacttcattgagccactcggccctgcattctgggagcgtaagtcatga

SEQ ID NO: 8 - protein, 408 aa, Corynebacterium glutamicum
MSNNNDKQYTTQELNAMSNEDLARLGTELDDVTIAYRKERFPIANDPAEKRAARAVTFWLVLGIIGGLGFLATYIFWPWEYKAHGDEGLLAYTLYTPMLGITSGLCILSLGFAVVLYVKKFIPEEIAVQRRHDGPSEEVDRRTIVALLNDSWQTSTLGRRKLIMGLAGGGAVLAGLTIIAPMGGMIKNPWNPKEGPMDVQGDGTLWTSGWTLVENDVKVYLGRDTAAIAESHTDATGEHWSTTGVSRLVRMRPEDLAAASMETVFPLPAEMVNDGAEYDPAKDVYEHQMHSVHGPRNAVMLIRLRTADAEKVIEREGQESFHYGDYYAYSKICTHIGCPTSLYEAQTNRILCPCHQSQFDALHYGKPVFGPAARALPQLPITVDEEGYLIAAGNFIEPLGPAFWERKS

SEQ ID NO: 9 - DNA, 1620 bp, Corynebacterium glutamicum
  CDS (1)..(1617)
atgagtctagctaccgtgggaaacaatcttgattcccgttacaccatggcgtcgggtatccgtcgccagatcaacaaggtcttcccaactcactggtccttcatgctcggcgagattgcgctttacagcttcatcgtcttgctgctgactggtgtctacctgaccctgttcttcgacccatcaatcaccaaggtcatttatgacggcggctacctcccactgaacggtgtggagatgtcccgtgcatacgcaactgcgttggatatttccttcgaggttcgcggtggtctgttcatccgccagatgcaccactgggcagccctgctgttcgttgtatccatgctggttcacatgctccgtattttcttcaccggtgcgttccgtcgcccacgtgaagcaaactggatcatcggtgttgttctgatcatcctgggtatggctgaaggcttcatgggttactccctgcctgatgacctgctctctggtgttggtcttcgaatcatgtccgccatcatcgttggtcttccgatcataggtacctggatgcactggctgatcttcggtggagacttcccatccgatctgatgctggaccgcttctacatcgcacacgttctaatcatcccagctatcctgcttggcttgatcgcagctcacctggcacttgtttggtaccagaagcacacccagttcccaggcgctggccgcactgagaacaacgtgatcggtatccgaatcatgcctctgttcgcagttaaggctgttgctttcggcctcatcgtcttcggtttcctcgcactgcttgctggtgtcaccaccattaacgcaatttggaatcttggaccgtacaacccttcacaggtgtctgctggttcccagcctgacgtttacatgctgtggacagatggtgctgctcgtgtcatgccggcatgggagctctacctcggtaactacactattccagcagtcttctgggttgctgtgatgctgggtatcctcgtggttctgcttgtgacttacccattcattgagcgtaagttcaccggcgacgatgcacaccacaacttgctgcagcgtcctcgcgatgttccagtccgcacctcactcggtgtcatggcgcttgtcttctacatcctgcttaccgtttctggtggtaacgatgtttacgcaatgcagttccatgtttcactgaacgcgatgacctggatcggtcgtatcggcctcatcgttggaccagctattgcatacttcatcacttaccgactgtgcatcggcttgcagcgctctgaccgcgaggtcctggagcacggcatcgagaccggtatcatcaagcagatgccaaatggtgccttcattgaagttcaccagccacttggcccagttgatgaccatggtcacccaatcccactgccatacgctggcgctgcggttccaaagcagatgaaccagcttggttacgctgaggttgaaacccgcggtggattcttcggacctgatccagaagacatccgtgcgaaggctaaggaaattgagcacgcaaaccacattgaggaagcgaacactcttcgtgcactcaacgaggcaaacattgagcgtgacaagaatgagggcaagaactag

SEQ ID NO: 10 - protein, 539 aa, Corynebacterium glutamicum
MSLATVGNNLDSRYTMASGIRRQINKVFPTHWSFMLGEIALYSFIVLLLTGVYLTLFFDPSITKVIYDGGYLPLNGVEMSRAYATALDISFEVRGGLFIRQMHHWAALLFVVSMLVHMLRIFFTGAFRRPREANWIIGVVLIILGMAEGFMGYSLPDDLLSGVGLRIMSAIIVGLPIIGTWMHWLIFGGDFPSDLMLDRFYIAHVLIIPAILLGLIAAHLALVWYQKHTQFPGAGRTENNVIGIRIMPLFAVKAVAFGLIVFGFLALLAGVTTINAIWNLGPYNPSQVSAGSQPDVYMLWTDGAARVMPAWELYLGNYTIPAVFWVAVMLGILVVLLVTYPFIERKFTGDDAHHNLLQRPRDVPVRTSLGVMALVFYILLTVSGGNDVYAMQFHVSLNAMTWIGRIGLIVGPAIAYFITYRLCIGLQRSDREVLEHGIETGIIKQMPNGAFIEVHQPLGPVDDHGHPIPLPYAGAAVPKQMNQLGYAEVETRGGFFGPDPEDIRAKAKEIEHANHIEEANTLRALNEANIERDKNEGKN

SEQ ID NO: 11 - DNA, 3549 bp, Corynebacterium glutamicum
  CDS (251)..(3307)
ccaatatatacagtgcaattcggatttccgttgggatctcagtcaagacacaggtagactgcccctgtattggtcgcgccctgtgcgacgccgactgagctttaaaagtgtttctcagttgacagacttggtttcttaattactaaaaaatcgatgtgtgttgctaactgggggtggcacgcacgttggcgttgttgtttggtgtggctccagagtaatccacaacgcgcaaaggggaactggagaacacgtgctcattctttttctcgcgctcactgcagccgcagtagtcgcccccatcctgatccgaactctcggtcgaccagcttttggtctgctggcgcttgtacctggcattggttttttctgggtgctttcggagttcatcaaaggcactttcaaggatggaggtgaactcctcctccactatgcctggatgccttcggctcacctcaatatcgatttccgtatggattccctcgcggcgctgttctcactcatcgtcttaggcgtgggcgccctagtgctgctgtactgctggggatattttgattccaacgcgggtcgcctcagtgcctttggtgctgaactggtggccttcgccatggcgatgtttggtcttgtcatttcagacaacatcctgctgatgtacgtcttctgggaaatcacctccgttttatccttcctcctggttggttattacggcgaacgcgcatcttcacgtcgctctgcaggtcaagccttgatggtgaccaccctgggtggattggccatgctggtgggcatcattttgatgggtacccaaactggcgtgtggcgattctctgagatccctgcctactcaagctcctgggcagatgtgccgtatatttccgctgctgctgcccttatcttggctggcgcactatccaaatcggctatcgcaccaacccacttctggcttcccggcgcgatggccgcaccaacgccggtgtctgcttacctgcactccgcagcgatggtgaaggcgggtatttaccttgtggctcgcctctctccagacctcaacgtagttggttcgtggtacctgatcatcatcccgttgggcatgttgaccatgctcatgggtggttggatggcgctgcgccaaaaggatctcaagctgatcctggcgtacggcacggtatcccagttgggcttcattatttccgtggtgggcattggtacccgcgaagctttgctggcaggtcttgcactgaccgttgcgcactccttgtttaaggcaacattgttcatgacagttggtgccattgaccacaccaccggaactcgtgatattcgtaaactctccggtctgtggcgtaaacaaccgatcctgttcgccgttgctgctgtttcggcggcgtccatggctggtattccgccactgtttggttttatcgccaaggaaacagcgctggataccgtgttgaatgagcagatgttgcatggcatgccaggtcgattgatgctggctggcatcgttttgggttccatcttcaccatggcatattcctgctacttcctgtacgaagcctttgccacgaagcactccaaattcccagaggccaacggtgtctcacctgcagtggaggcaatgcatccggtgaagtttaagctgtggatcgcacctgtcatcctggctattttgaccgtagtgtttggtgttttccccaagccagtgtcggaagcaattgtcacgcatcttgataacgtcacgccatcgcttgatgatgtccacaccaaactggccttgtggcatggtctgaatctaccgctgctgctgtctgtggtgatcatcatttccggattcatcatcttctgggagcgagacaccgtcgaacgtttgcgccctaacaccgcagcgtttggcagtgccgataccgcctacgacgccattcttgatgcactgcgtgtgctctcccaccgcctgactgcatccacccagcgtggttctttgaccctgaacgtcggtgtgatcttcttcgtcctcacgattgttccgctgatcgctttgatcactggcgaacaaagcgatgtccgcatggagctgtgggatagccctattcagggcttcatcgcggccatcattatcgtcgttgcgattgtggcaaccaccatggataaccgtttgtctgcgctgattttggtgggtgtgacaggttatggcattgccgttatcttcgcgctacatggcgcaccggacttggcgctaacccaggtgctggtggagaccatcgtcatggtggtattcatgctggtgctgcgtaaaatgccgacagaagttgcgtggaaggcagaacctaaacagtctcgcgtgcgagcgtggcttgctggcgccaccggattgtccgttgttattgtcaccatttttgccatgaatgctcgcaccactgaaccgatctctgtatacatgcaggatctggcctatgagatcggacatggcgcaaacaccgtcaacgtactgctcgtagacctgcgtggttttgataccttcggtgaaatttccgtccttgtgatcgcggcaaccggtatcgcctccctggtctaccgaaaccgcagcttccgcaaggattctcgcagaccaaccctggctaccactggtcgccgttggttggctgctgctgttgataccgaaagggcgcagaaccgctcgctgatggttgatgtggcaacgcgcatcctcttccctgccatgatcatgttgtctgtgtacttcttcttcgccggacacaacgcgccgggcggcggattcgccggcggccttgttgcctccttggcgttcgccttgcgctaccttgccggtggacgtgaagaacttgaagaagcgttgcctatcgacgccggccgtatcttgggaactggactatttgtttctgcaactgcagtgctgtggcccatggttcttcttggtgaaccaccgctgacctcccatatttgggatctcacactgccacttatcggtgagattcacattgcatccgcgctgctctttgaccttggtgtctacctgatcgtcatcggtttgaccatgcacattctcaacagtttgggcggccagctcgaccgcgatgaggaaatgcgtaagcagcgtgcgcgcgaccgagctcgacgcttggcgcgcaaccagcgtcgagaagcagcaaccgtcggcgcacgcaggtcgaacgagaaatcgacacgccaaatgccgacgattcggcctccaggggcagacacagaatcggtggagcagaacggtgagaaccagacgtcgataagcacaaagcgtttaaagcaggaaggaaaataacacatggtagccaaccttttcctgctcctagctgctggaactctcatttctgcgggtgtgtatctgctgcttgatcgcgcgatgaccaaaatgatcatgggtctcatgctgatcggcaacggagccaacctgctgattttggtcgctggaggttccgctggatcgccaccgattctggggcgtgaaagcgaaatctacggcgacaaaaccgctgatccgctagcccaagccatgatcct

SEQ ID NO: 12 - protein, 1019 aa, Corynebacterium glutamicum
MLILFLALTAAAVVAPILIRTLGRPAFGLLALVPGIGFFWVLSEFIKGTFKDGGELLLHYAWMPSAHLNIDFRMDSLAALFSLIVLGVGALVLLYCWGYFDSNAGRLSAFGAELVAFAMAMFGLVISDNILLMYVFWEITSVLSFLLVGYYGERASSRRSAGQALMVTTLGGLAMLVGIILMGTQTGVWRFSEIPAYSSSWADVPYISAAAALILAGALSKSAIAPTHFWLPGAMAAPTPVSAYLHSAAMVKAGIYLVARLSPDLNVVGSWYLIIIPLGMLTMLMGGWMALRQKDLKLILAYGTVSQLGFIISVVGIGTREALLAGLALTVAHSLFKATLFMTVGAIDHTTGTRDIRKLSGLWRKQPILFAVAAVSAASMAGIPPLFGFIAKETALDTVLNEQMLHGMPGRLMLAGIVLGSIFTMAYSCYFLYEAFATKHSKFPEANGVSPAVEAMHPVKFKLWIAPVILAILTVVFGVFPKPVSEAIVTHLDNVTPSLDDVHTKLALWHGLNLPLLLSVVIIISGFIIFWERDTVERLRPNTAAFGSADTAYDAILDALRVLSHRLTASTQRGSLTLNVGVIFFVLTIVPLIALITGEQSDVRMELWDSPIQGFIAAIIIVVAIVATTMDNRLSALILVGVTGYGIAVIFALHGAPDLALTQVLVETIVMVVFMLVLRKMPTEVAWKAEPKQSRVRAWLAGATGLSVVIVTIFAMNARTTEPISVYMQDLAYEIGHGANTVNVLLVDLRGFDTFGEISVLVIAATGIASLVYRNRSFRKDSRRPTLATTGRRWLAAAVDTERAQNRSLMVDVATRILFPAMIMLSVYFFFAGHNAPGGGFAGGLVASLAFALRYLAGGREELEEALPIDAGRILGTGLFVSATAVLWPMVLLGEPPLTSHIWDLTLPLIGEIHIASALLFDLGVYLIVIGLTMHILNSLGGQLDRDEEMRKQRARDRARRLARNQRREAATVGARRSNEKSTRQMPTIRPPGADTESVEQNGENQTSISTKRLKQEGK

SEQ ID NO: 13 - DNA, 2121 bp, Corynebacterium glutamicum
  CDS (228)..(1916)
cgcgttcatgctgtcgcttgcctaccgtcaataccgttaccgcaccgaggacttcattgaagatgacaccgaggacgttgcaatcaccgtccgccccagttttgcgtctgctgcacctgaccacgatgcatctgacgacccagaaactggtcgcatgacctcagacggcgacgatttcggcccagaatccttcgaagcaccactgaagggagataaggatgactagtttgtacgaaactctcgtcccgttgatcccttatatggtcccgctgcccatcattttgcctgcggttgcggcggcgctggcattgattttgtcgaaatatctgacagcgcagcgcaccattactttgtctgttttggcgttcctcattggtcttaacgccaccatgctgtacgtggtggatcgcgaaggcattcagactttgcagttgggtggctgggatgccccgatcggaatcaccctggtggccgaccgactgtctgtttccatgctgacggtgagttccatcgtgctgttttctgtcatgtggtacgcgatcagccagggtattcgcgacggcggcaaggacgaacctgttgcagtgttcctgcctacttacctgctgctctcgatgggcgtgaacctggcgttcctcgctggcgacctgtttaacctctacgttggtttcgaagtgctgctggtggcgtcatacgtgctgctcaccttgggtgcatcgccggcacgtgtacgttccggcgtgggttacgtgatggtgtccatggcgtcatcgatggtgttcctgtttggactcgcaatggtttacgcctcagtgggcacgttgaacatggctcacgttggcctacgcatggaagatgttccgtctggaactcgctccgcgatcttcgcagtgttgctcgtggcattcggtattaaagctgccgtgttccccctagattcctggctgccggactcctaccccaccgcgccatcgctggtcaccgcggtgttcgcaggtctgttgaccaaggtgggtgtgtattccatcattcgagcacgctcgattattttcaccgatggatcccttgacaccatgctgatgtgggtggcactcgccaccatgctcattggtattttgggcgcgatggcgcaaaacgatatcaaacgtttgttgtcatttactctggtcagccacatcggctacatgatcttcggcgtagcccttggatctgcacagggtttgtctggtgcgatcttctacgcaatccaccacattctggttcagacttccctgttcctggtggtcggtctggtggaacgccaagccggatcctcctcgctgcgacgccttggatccctggcatatatctccccacttcttgcgattttgtacttcatccccgccatcaacctgggtggtatcccaccgttctccggcttcctgggcaagatcatgctcatcgaagccggcgccgaagatggcagttggctggcatgggtccttatcgcaggcgccgttgtcacctcactgctcaccttgtacaccatggttctggtctggtccaaggccttctggcgcgaccgtaaagacgcccccgatggagcaaccgcactagcaagacccgcacctttggtagatatccaagacgaagtcgccgttaaagaccgcaacgatgtcggacggatgccttggggcatggtcttctccactgccctgttggtttccgcatcccttgctgtatccgtgctcgcaggaccactgtcatctattactggacgcgccgccgaatccgcacaagatgtcaacatctaccgcgccgcagtactcggccccaactacctcgacccatcacgcacactcgagatggagcgttacgacgccaaccgcgatgacatcaaccaccgcgtcgacaccaatggaacggaggaccaaccatgatcagtggattcaaacgacgcttccgccccgtcttcatcatcggcctcaccctgatgtgggtcatgctcatggcagaattcacctgggcaaacttcgtcggcggtttcctcgtagctagcgcaatcgtgctgttcctcccacttccagccatgccgattgagaacatctccatccgctggggatcactcatcctgctgatcct

SEQ ID NO: 14 - protein, 563 aa, Corynebacterium glutamicum
MYETLVPLIPYMVPLPIILPAVAAALALILSKYLTAQRTITLSVLAFLIGLNATMLYVVDREGIQTLQLGGWDAPIGITLVADRLSVSMLTVSSIVLFSVMWYAISQGIRDGGKDEPVAVFLPTYLLLSMGVNLAFLAGDLFNLYVGFEVLLVASYVLLTLGASPARVRSGVGYVMVSMASSMVFLFGLAMVYASVGTLNMAHVGLRMEDVPSGTRSAIFAVLLVAFGIKAAVFPLDSWLPDSYPTAPSLVTAVFAGLLTKVGVYSIIRARSIIFTDGSLDTMLMWVALATMLIGILGAMAQNDIKRLLSFTLVSHIGYMIFGVALGSAQGLSGAIFYAIHHILVQTSLFLVVGLVERQAGSSSLRRLGSLAYISPLLAILYFIPAINLGGIPPFSGFLGKIMLIEAGAEDGSWLAWVLIAGAVVTSLLTLYTMVLVWSKAFWRDRKDAPDGATALARPAPLVDIQDEVAVKDRNDVGRMPWGMVFSTALLVSASLAVSVLAGPLSSITGRAAESAQDVNIYRAAVLGPNYLDPSRTLEMERYDANRDDINHRVDTNGTEDQP

SEQ ID NO: 15 - DNA, 3500 bp, Corynebacterium glutamicum
  CDS (239)..(3256)
atgagcgggaccaatagcaggagggcagtcaagatgtttcttgatcgagaagcagcggcagacatagtgtgcaatctagttattgtttcgaggtttagcagggaggcgcaccgaaatgtgctcttgctaactcatcaagtagttttgtaaatagtcaatttaaagggtaaatgccctcccgtgtcactgaaatcgttttggcggtggcgagggttctatgggcgattagggataatgcatgagtttgctatttgttgtggcgcttgctgtgatctcagtatttttggcgccaatttcggtcaaggtgattgatcgaaaagctggatggcctttggctgtaatctttgcagtcgccgcatattttttggtgcgtgaggctggtccaattttggatggtcaggcgctgacctgggatatcacgtgggtgagggatattctcggctcgggtgtcgacgtcaagtttgccttgcgcgctgatgcgttgagtttgttctttgcactgttggcgctggttatcggcgctgtggtgtttgtgtattcggcggaatatttgccgcggaagaagggcaacacgagtttttacaccatcatgacggcgtttaccgctgccattttgctgctcgtgcttgccgatgatgtctttgtgttgttcgtcggctgggaactggtctcgcttgcgtcattcatgttgattgcgcgttcgggttcttctggtgaatctggttcgatccgcacgttgattttgacgtttttcggcggcctcacgctgcttactgcggtggcgattgctgcgacccaagctggcaccaccagcctcgatggtattttgcactctgatttctgggcggagaagccagtgctcacgggtgttattgcggtgctgattgcaatgtccgcgttcactaagtccgcacagttcccgttccacttctggctgcctgaggcgatggctgcggccaccccagtgtcggcgttcctgcacgctgcggccgtggtcaaggcgggtatttacctgttgctgcgctttagcattgtgttccatgatgttgcggtctggaattggttgctgattatcgtcggcatgggtacggccatcatgtcggcgtatttcgcggtgcagaagaccgatctgaagaagctcacggcatattccacggtgtcgcatttgggttggatcgtagcgaccatcggcgtgggcactcctttcgcgctcggcgctgccattgtgcacacgctcagccacgcgctgtttaagtcctcgttgttcatgctcattggcgtgattgatcaccagactggcacgcgcgatattcgtcgcctcggtttcctggtcaagaagatgccgttcacgtttgtgtctgtattaataggtgcgttgtcgatggcatcggttccgccgttgctcggcttcgtgtccaaagaaggcatgatcacagcgttcatggacgcccccatcggcaactcctatgttgtattactgctggtcggcgcagcaatcggcgcggtcctaaccttcacatactccgcgaaactcgtgctcggcgcattcgtcgacggcccacgcgacatgtcacacgtcaaggaagcccccgtctccctctggcttccggccgccctgcctggacttatgtctctgccactagtcctagtactttcgcttttcgacgcccccgtctccgccgcagccacctccgccgcgggggaagcggcgcacatgcacctggcattgtggcacggcatcaacaccccactgttgatttccttgggtgtgctggtggccggaatccttggtgtgctgttccgcaaagagctgtggaaaatcgccgagaccagccctttccccatcgccacaggcaacgacatcctatcgatgctggtttaccgagccaacttgctgggtaaattcttcggtcgcatggctgattcgatgagcccacgcaggcacttggtcagcctcatcgtgctgctctgggcgctggctgcttttgccaccattcacccctcggttcagcttgcaccaaagcaaccgggaattgatcgttggatcgacctcattccgcttgccatcatcgcgctatctgtcttcggcctgctcaccacccgaaaccgcctcagcgcagccgtgcttgtgggtaccgttggtgtgggtgtttccttccagatgctacttctgggcgctcccgatgttgcacttacccagttcctggtagaaggcctcgtcgtggtaatcatcatgatggttgtccggcaccagcctgccaacttcaagcgcatcaagcccagcagaaggcgcagcaccgttcttgtcgccgtccttgctgccttcgccgcattcatggcggtgtggggattgcttggccgtcacgaacgttctgagctggccatgtggtacctcaaccaaggtccagagatcacctctggcgccaacgtggtgaacaccatcctcgtggaattccgtgcactggatacgttgggcgagctctccgtgcttggcatggcagctgtcgtcatcggtgcgatggtggcttccatgcctcgtcatccgtttgccaagggcacccaccctcgcccctttggccaatcacagttgaactccattccgctgcgcatgctgcttaaggtgctggttccagcgctatgcttcttgagcttcatggtgttcatgcgtggacacaatgatccggaaggcggtttcatcgcagccctaattgccggtggcgcgctgatgctcctgtacctgtccaaggccaaagatggccgcattttccgcccgaatgttcctttcattctcactggtgcgggcatcttgatggcagtgttctcgggcgtactgggactcacccacggttctttcctgtacgccatccacttcaacttcgtaggccagcactggaccacctcgatgatcttcgacctcggcgtgtacctggccgtgttgggcatggtgtccatggcaatcaacggcctgggcggatacctgcgcccaggtaccgacattgcagatctggactacgcccgccgaagtggcccactgccagcaacgccaacggttgaacccgaaccagaaggcgatgaagactggcccgaacccatcaaccccgcaggcgatacaaagagaggcaaaccgatgattctcgcactgacaagtcgcgatacttttcgggtggagggtgtctaccttcaattaagcaaacgcgggaaatggtgcgacattcgttcttcggcaatgtcacttgatcggccaacgaagcgaacttgaccatcctgtacgccggtgtgccccacgtggcgcggcaaaagccttcccagacaggaccccggcttaccgacgccgccgatccactcccacaggccttcgtcctcaccgccatcgtcatcgcgatggccaccacaaccatcatgttggccttggcagcactgggacgcagcgacgacacccggtccatcgaaccagatgacgatcaatcgcctttgactactagcgctcgttcagtcaccaa

SEQ ID NO: 16 - protein, 1006 aa, Corynebacterium glutamicum
MSLLFVVALAVISVFLAPISVKVIDRKAGWPLAVIFAVAAYFLVREAGPILDGQALTWDITWVRDILGSGVDVKFALRADALSLFFALLALVIGAVVFVYSAEYLPRKKGNTSFYTIMTAFTAAILLLVLADDVFVLFVGWELVSLASFMLIARSGSSGESGSIRTLILTFFGGLTLLTAVAIAATQAGTTSLDGILHSDFWAEKPVLTGVIAVLIAMSAFTKSAQFPFHFWLPEAMAAATPVSAFLHAAAVVKAGIYLLLRFSIVFHDVAVWNWLLIIVGMGTAIMSAYFAVQKTDLKKLTAYSTVSHLGWIVATIGVGTPFALGAAIVHTLSHALFKSSLFMLIGVIDHQTGTRDIRRLGFLVKKMPFTFVSVLIGALSMASVPPLLGFVSKEGMITAFMDAPIGNSYVVLLLVGAAIGAVLTFTYSAKLVLGAFVDGPRDMSHVKEAPVSLWLPAALPGLMSLPLVLVLSLFDAPVSAAATSAAGEAAHMHLALWHGINTPLLISLGVLVAGILGVLFRKELWKIAETSPFPIATGNDILSMLVYRANLLGKFFGRMADSMSPRRHLVSLIVLLWALAAFATIHPSVQLAPKQPGIDRWIDLIPLAIIALSVFGLLTTRNRLSAAVLVGTVGVGVSFQMLLLGAPDVALTQFLVEGLVVVIIMMVVRHQPANFKRIKPSRRRSTVLVAVLAAFAAFMAVWGLLGRHERSELAMWYLNQGPEITSGANVVNTILVEFRALDTLGELSVLGMAAVVIGAMVASMPRHPFAKGTHPRPFGQSQLNSIPLRMLLKVLVPALCFLSFMVFMRGHNDPEGGFIAALIAGGALMLLYLSKAKDGRIFRPNVPFILTGAGILMAVFSGVLGLTHGSFLYAIHFNFVGQHWTTSMIFDLGVYLAVLGMVSMAINGLGGYLRPGTDIADLDYARRSGPLPATPTVEPEPEGDEDWPEPINPAGDTKRGKPMILALTSRDTFRVEGVYLQLSKRGKWCDIRSSAMSLDRPTKRT

SEQ ID NO: 17 - DNA, 23 bp, Corynebacterium glutamicum
  misc_feature (14)..(14): n= inosine
ttgttctttctggncactccnga

SEQ ID NO: 18 - DNA, 24 bp, Corynebacterium glutamicum
  misc_feature (3)..(3): n=inosine
gtntgggcncactcactatgttct

SEQ ID NO: 19 - DNA, 22 bp, Corynebacterium glutamicum
  misc_feature (14)..(14): n=inosine
aacatagtgagtgngcccanac

SEQ ID NO: 20 - DNA, 21 bp, Corynebacterium glutamicum
  misc_feature (19)..(19): n=inosine
acagtaagtgagaaagtgngc

SEQ ID NO: 21 - DNA, 27 bp, Corynebacterium glutamicum
  misc_feature (3)..(3): n-inosine
acnggnttctcactggncttncactgt

SEQ ID NO: 22 - DNA, 26 bp, Corynebacterium glutamicum
  misc_feature (22)..(22): n-inosine
tacttacttggcactttctgtngact

SEQ ID NO: 23 - DNA, 26 bp, Corynebacterium glutamicum
  misc_feature (5)..(5): n=inosine
agtcnacagaaagtgccaagtaagta

SEQ ID NO: 24 - DNA, 24 bp, Corynebacterium glutamicum
  misc_feature (13)..(13): n-inosine
agttagtgagcangcatngcagca

SEQ ID NO: 25 - DNA, 23 bp, Corynebacterium glutamicum
  misc_feature (17)..(17): n=inosine
gcatcttgagtgagcanggagca

SEQ ID NO: 26 - DNA, 30 bp, Corynebacterium glutamicum
actgtcgacggctgtagttaactgcaaccg

SEQ ID NO: 27 - DNA, 42 bp, Corynebacterium glutamicum
cccatccactaaacttaaacaaggcgccacagcggtcatagg

SEQ ID NO: 28 - DNA, 42 bp, Corynebacterium glutamicum
tgtttaagtttagtggatgggccagaattgggtaccgcccca

SEQ ID NO: 29 - DNA, 21 bp, Corynebacterium glutamicum
actgtcgacggtctcgacagg

SEQ ID NO: 30 - DNA, 28 bp, Corynebacterium glutamicum
actgtcgacctcaacgtgccctacgcac

SEQ ID NO: 31 - DNA, 42 bp, Corynebacterium glutamicum
cccatccactaaacttaaacatggggtctgcgggttggttcc

SEQ ID NO: 32 - DNA, 44 bp, Corynebacterium glutamicum
tgtttaagtttagtggatggggaggcaaacattgagcgtgacaa

SEQ ID NO: 33 - DNA, 30 bp, Corynebacterium glutamicum
tgagtcgacctgcaatttcaggaaacttcc

SEQ ID NO: 34 - DNA, 27 bp, Corynebacterium glutamicum
acttctagatagggttgacattttgtc

SEQ ID NO: 35 - DNA, 57 bp, Corynebacterium glutamicum
agtgtcgacctaatggtgatggtgatggtgagctgcgttcttgccctcattcttgtc

(19) United States
(12) Patent Application Publication
(10) Pub. No.: US 2004/0014180 A1
(43) Pub. Date: Jan. 22, 2004

(54) Method for the microbial production of metabolic products, polynucleotides from coryneform bacteria and use thereof

(76) Inventors: Michael Bott, Julich (DE); Axel Niebisch, Julich (DE); Brigitte Bathe, Salzkotten (DE); Achim Marx, Bielefeld (DE); Thomas Hermann, Blelefeld (DE)

Correspondence Address: SMITH, GAMBRELL & RUSSELL, LLP, 1850 M STREET, N.W., SUITE 800, WASHINGTON, DC, 20036, US

(21) Appl. No.: 10/380,055

(22) PCT Filed: Sep. 8, 2001
(86) PCT No.: PCT/DE01/03485

(51) Int. Cl. C12P 19/34; C12P 13/04; C12P 13/24; C12P 13/22; C12P 13/20; C12P 13/14; C12P 7/60; C12P 7/56; C12P 7/54; C12P 7/06; C12N 1/21

(52) U.S. Cl. 435/91.1; 435/106; 435/107; 435/108; 435/109; 435/110; 435/138; 435/139; 435/140; 435/161; 435/252.3

(57) ABSTRACT

The invention relates to a method for the microbial production of metabolic products, polynucleotides from coryne-form bacteria and use thereof. According to the invention, by means of said method and polynucleotides it is possible to influence the synthesis of ATP in a controlled manner and also to control the synthesis of metabolic products. The invention relates to genes from Corynebacterium glutamicum coding for cytochrome aa₃ oxidase and the cytochrome bc₁ complex. The monocistronic ctaD gene codes for a 65 kDa protein, the primary structure of which displays all the typical properties of the sub-unit I of cytochrome aa₃ oxidase. The genes which code for the sub-unit III of the cytochrome aa₃ (ctaE) and the three characteristic sub-units of the cytochrome bc₁ complex (qcrABC) are arranged in a group with the sequence ctaE-qcrCAB. An analysis of the derived primary structure shows a sequence with unusual properties: (i) cytochrome C₁ (QcrC, 30 kDa) contains two Cys-X—X-Cys-His groups for the covalent bonding of haeme, which means that said protein is a di-haeme cytochrome of the c type; (ii) the “Rieske” iron-sulphur protein (QcrA, 45 kDa) presumably contains three trans-membrane helices in the N-terminal region; (iii) cytochromeb (QcrB, 60 kDa) contains a C-terminal extension of 120 amino acids, along with the conserved region with 8 trans-membrane helices, presumably localised in the cytoplasma. The electron transfer from the cytochrome bc₁ complex to the cytochrome aa₃ terminal oxidase does not involve additional cytochrome c.

The invention concerns a method for microbial production of metabolic products, polynucleotides from coryneform bacteria and their use.

Corynebacterium glutamicum (C. glutamicum), a gram positive soil bacterium with a high G+C content (54 mol %), is used for the industrial production of amino acids, especially for the production of L-glutamate and L-lysine. The synthesis pathway of these amino acids, the central metabolism which makes available the relevant precursors, and the carbon flux and nitrogen flux of this organism have been intensively studied (Sahm et al. 1995; Eggeling and Sahm 1999).

Less investigated is the composition and efficiency of the respiratory chain with coryneform bacteria. Organisms that breathe synthesize ATP by oxidative phosphorylation. Here the reduction equivalents (H or electrons) that arise in the oxidation of substrates are transferred to the membrane-associated respiratory chain and in the end the electrons are carried to oxygen or other terminal electron acceptors. Energy generating and energy consuming metabolic pathways are coupled to each other via ATP and the electrochemical proton potential or the electrochemical sodium ion potential as universal cellular energy forms. The synthesis of ATP can take place either via substrate stepwise phosphorylation or by electron transport phosphorylation. The building of the electrochemical proton potential takes place through the respiratory chain or the hydrolysis of ATP by the membrane-based F₀F₁-ATP synthase. The components of the respiratory chain are enzymes, which as a rule contain covalently or non-covalently bound low-molecular groups, for example flavins (flavoproteins), iron-sulfur centers (iron-sulfur proteins) and heme groups (cytochromes). Quinones, i.e., low-molecular, membrane-based electron and proton carriers such as a ubiquinone and menaquinone, are another essential component of respiratory chains. In C. glutamicum only menaquinone (MK) is known with regard to the quinones of the respiratory chain. Besides the predominant form MKH₂-9, i.e., the reduced quinone with nine isoprene units, there are also small amounts of MK-9 and MKH₂-8 (Collins et al. 1977). In the case of C. glutamicum ATCC 13032 it was possible to detect, by means of reduced minus oxidized difference spectra, cytochromes of a-type (absorption maximum at about 600 nm), b-type (absorption maximum at about 560 nm) and c-type (absorption maximum about 550 nm) (Trutko et al. 1982). The CO reduced minus reduced difference spectrum showed a peak at 427 nm and another at 443 nm, which points to a terminal oxidase of cytochrome aa₃-type. Isolated membranes showed NADH, NADPH, succinate and lactate oxidase activity, with NADH exhibiting a 5 to 8 times higher rate than the other substrates (Trutko et al. 1982; Matsushita et al. 1998). The oxidase activity with the electron donor tetramethyl-p-phenylenediamine (TMPD) was approximately 50% of the activity obtained with NADH, whereas the rate of the cytochrome c oxidation was only 1% of the oxidation rate of NADH. With the TMPD oxidase system the Kₘ value for oxygen was 56 μM, whereas the NADH oxidation system had two Kₘ values of 18 μM and 48 μM. The TMPD oxidase activity that is associated with the cytochrome a fraction of solubilized membrane proteins was completely inhibited by 0.1 mM cyanide. The NADH oxidase activity, which was obtained only with membranes, but not with solublized membrane proteins, was only inhibited by 50% by 0.1 mM cyanide and an activity of 20% was still observed after treatment with ˜5 mM cyanide (Matsushita et al. 1998). These results can suggest the presence of two terminal oxidases: (i) a cytochrome aa₃ terminal oxidase, which is inhibited by micromolar concentrations of cyanide, has a low TMPD:cytochrome c oxidase activity, and has a Kₘ of about 50 μm for oxygen; (ii) an alternative okidase that is not inhibited until millimolar cyanide concentrations are reached, does not oxidize TMPD and has a Kₘ value for oxygen of about 20 μM.

Although no cytochrome d specific peak was observed in the redox difference spectra (Trutko et al. 1982, Matsushita et al. 1998), the results of Kusumoto et al. (2000) unambiguously point to the alternative oxidase activity at least partially deriving from a menaquinone oxidase of bd type. Besides the peaks at 550, 560 and 603 mm redox difference spectra showed a peak at 630 nm, which is characteristic for cytochrome d. The purified cytochrome bd oxidase consists of two polypeptides with molecular weights of 556 and 42 kDa, is activated with incubation with menaquinone, and shows a Ki value of 5.3 mM for cyanide. The cydAB genes code for the subunits I and II of the bd terminal oxidases and form an operon (Kusumoto et al. 2000).

According to these data, C. glutamicum has a branched respiratory chain with at least two branches. Common to both is the initial transfer of reduction equivalents of NADH, succinate, lactate or malate (Molenaar et al. 1998) to menaquinone. From there they are transferred either to the cytochrome bc₁ complex, whose existence is suggested by the c type cytochromes, and then to the terminal cytochrome aa₃ oxidase. Alternatively, the reduction equivalents can be transferred from reduced menaquinone to the terminal cytochrome bd oxidase. In order to investigate the different functions of these two pathways and their effect on amino acid production, it is necessary to use microorganism strains in which either the cytochrome aa₃ oxidase or the cytochrome bd oxidase is missing. The inventors were able to identify genes that code for the subunits I and III of the cytochrome aa₃ oxidase and for the cytochrome bc₁ complex. The ATP produced by the reactions of the respiratory chain among other ways, is the universal carrier of chemical energy between energy-producing and energy-consuming reactions and thus serves heterogeneous processes like synthesis of building blocks and macromolecules. Energy-producing and energy-consuming metabolic pathways can, among other methods, be controlled via the energy budget of the cell or its ATP content.

It is therefore a task of the invention to find substances and to create a method with which the metabolism of cells can be regulated. It is also a task of the invention to find substances in which RNA, cDNA and DNA sequences are found that can be used for isolation of nucleic acids, polynucleotides or genes.

Based on the generic part of claim 1, the task is solved in accordance with the invention with the characteristics given in the characterizing part of claim 1. In addition, the task based on the generic part of claim 9 is solved in accordance with the invention with the characteristics given in the characteristic part of claim 9. Furthermore, the task based on the generic part of claim 15 is solved in accordance with the invention with the characteristics given in the characterizing part of claim 15.

Through the polynucleotides in accordance with the invention and the method in accordance with the invention it is now possible to have a targeted effect on the synthesis of ATP through electron transport phosphorylation and on the synthesis of the electrochemical proton potential through the respiratory chain and thereby to enable control of the synthesis of metabolic products. For example, the microbial production of metabolic products such as amino acids (L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan, L-arginine), organic acids (acetic acid, citric acid, isocitric acid, lactic acid, succinic acid, fumaric acid, ketoglutaric acid, pyrotartaric acid, malic acid), vitamins, nucleosides, nucleotides and mono- or polyhydric alcohols can be positively influenced by establishing a suitable energy charge. In addition, the polynucleotides in accordance with the invention can be used as hybridization probes for detection of RNA, cDNA and DNA and for isolation of nucleic acids, polynucleotides or genes.

Advantageous embodiments are given in the subordinate claims.

The figures show as examples the components that participate in the respiratory chain with the pertinent genes as well as experimental results of the methods conducted using the polynucleotides in accordance with the invention:

Here:

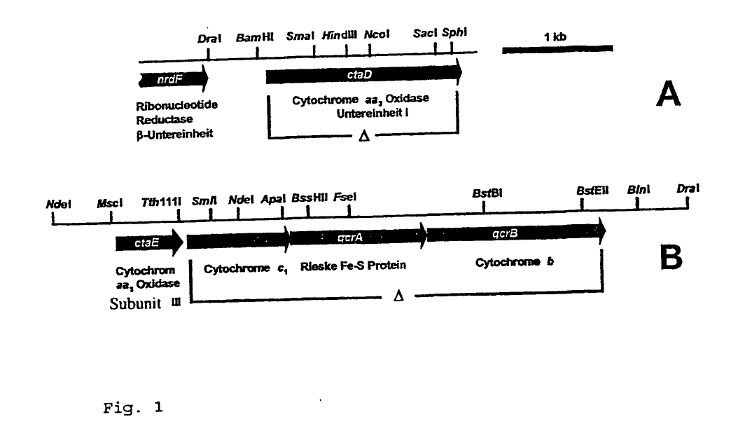

FIG. 1 is a representation of the genome region of Corynebacterium glutamicum with the ctaD gene region (A) and the ctaE-qcrCAB gene region (B). The DNA regions that were deleted in strain 13032ΔctaD or in strain 13032Δqcr are marked.

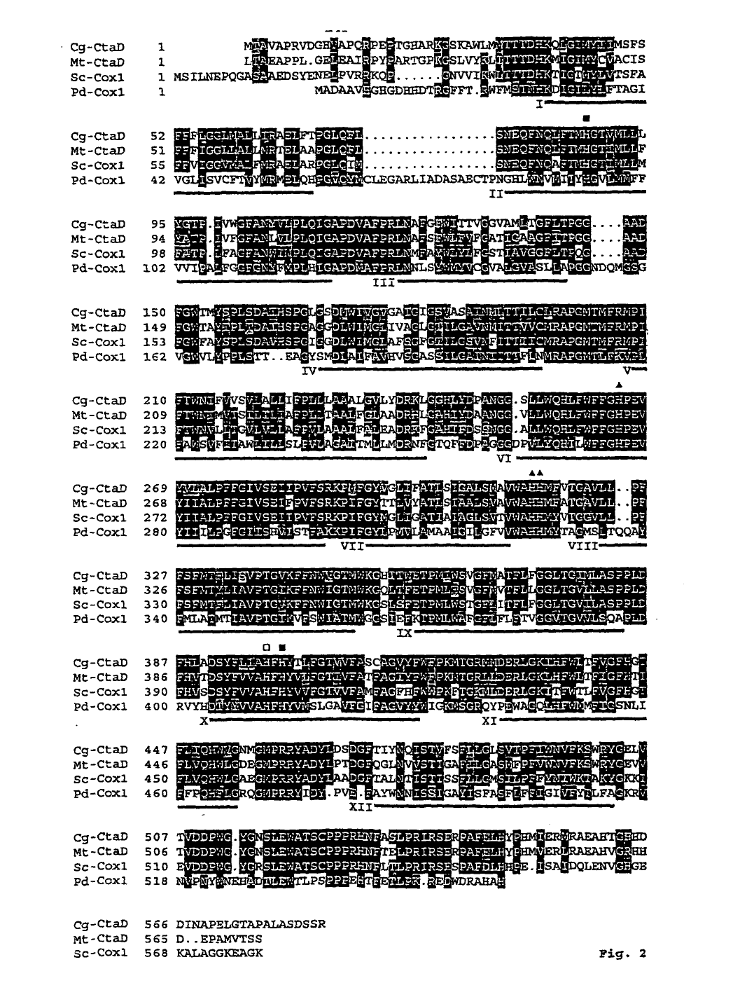

FIG. 2 is a sequence comparison of subunit I of cytochrome aa₃ oxidase from C. glutamicum (this invention), M. tuberculosis (Col et al. 1998), Streptomyces coelicolor and P. denitrificans (Raitio et al. 1990). Amino acids that are identical in at least three of the four sequences were indicated with a black background, while conserved amino acid exchanges were marked with a gray background. The 12 transmembrane helices that were identified in the crystal structure of cytochrome aa₃ oxidase from P. denitrificans were marked by black bars (Iwata et al. 1995). The histidine residue that serve as ligands for Cu_{B}, heme a and heme a₃ are represented by triangles, black squares and open squares.

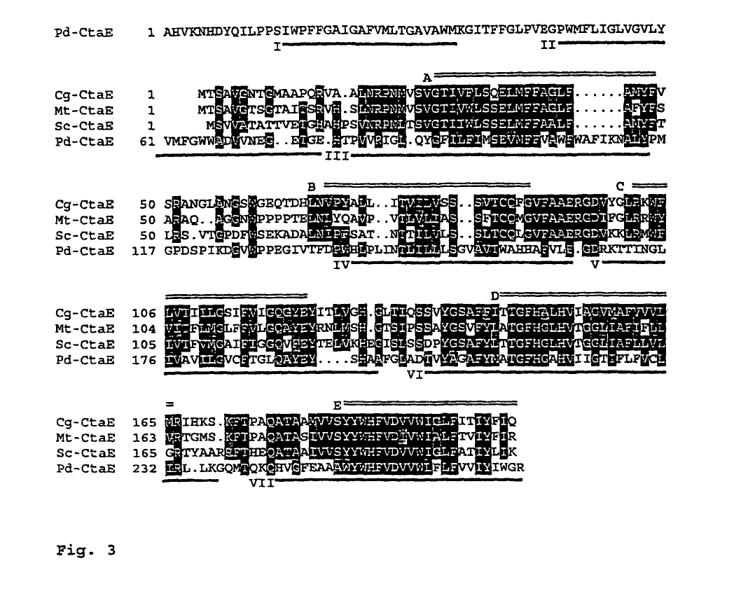

FIG. 3 is a sequence comparison of subunit II from cytochrome aa₃ oxidase from C. glutamicum (this invention), M. tuberculosis (Cole et al. 1998), S. coelicolor and P. denitrificans (Raitio et al. 1990). Amino acids that are identical in at least three of the four sequences were marked with a black background, conserved amino acid exchanges were marked with a gray background. The transmembrane helices I-VII that were identified in the crystal structure of the cytochrome aa₃ oxidase of P. denitrificans (Iwata et al. 1995) are marked by black bars. The transmembrane helices predicted for CtaE from C. glutamicum (A-E) are marked by double lines.

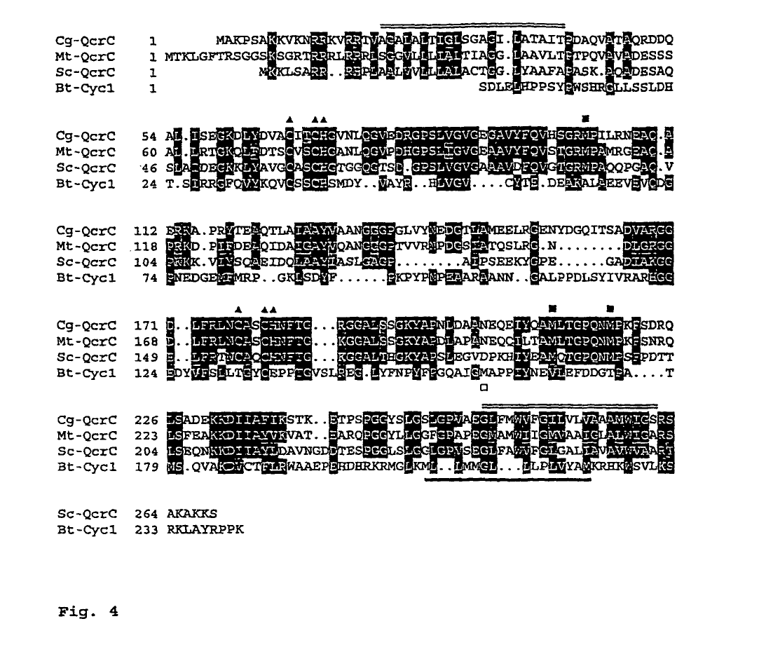

FIG. 4 is a sequence comparison of cytochrome c₁ subunit of the bc₁ complex from C. glutamicum (this invention), M. tuberculosis (Cole et al. 1998), S. coelicolor and cow (Wakabayashi et al. 1982). Amino acids that are identical in at least three of the four sequences were marked with a black background, conserved amino acid exchanges were marked with a gray background. The transmembrane helix that was identified in the crystal structure of bovine cytochrome c₁ was (Xia et al. 1997; Zhang et al. 1998; Iwata et al. 1998) is marked with a black bar. The two possible transmembrane helices that were predicted for QcrC from C. glutamicum are represented by double lines. The two heme binding motifs are indicated by filled triangles, potential methionine ligands of the QcrC heme-iron atom by filled squares, and the detected methionine ligand of the bovine cytochrome c₁ by an open square.

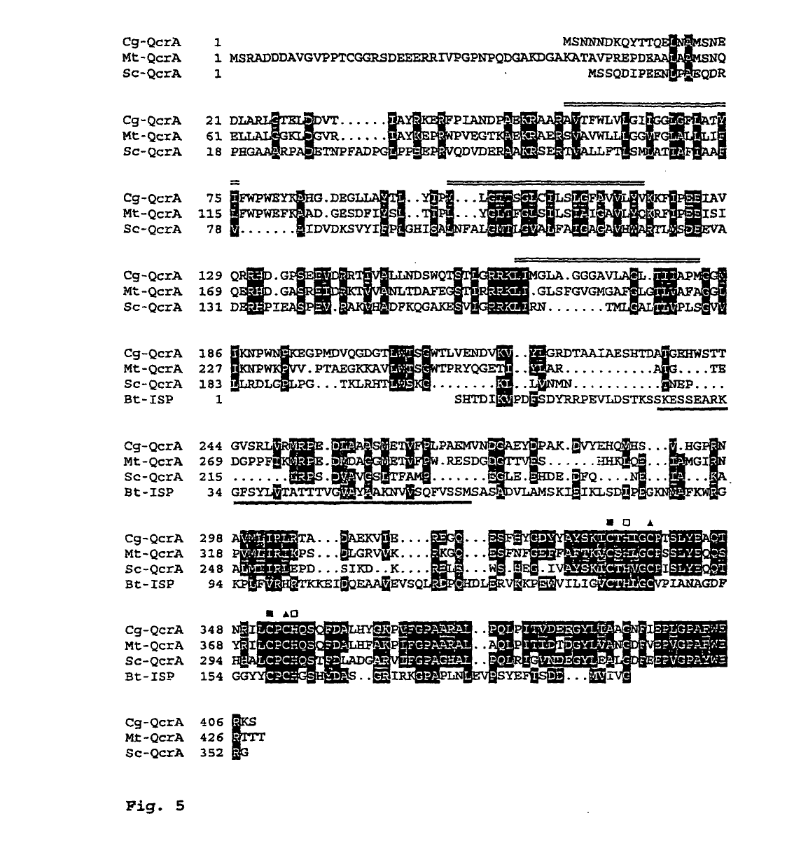

FIG. 5 is a sequence comparison of the iron-sulfur protein subunit of the bc₁ complex from C. glutamicum (this invention), M. tuberculosis (Cole et al. 1998), S. coelicolor and cow (Schagger et al. 1987). Amino acids that are identical in at least three of the four sequences were identified with a black background, conserved amino acid exchanges were marked with a gray background. The transmembrane helix that was identified in the crystal structure of the iron-sulfur protein from cow (Xia et al 1997; Zhang et al. 1998; Iwata et al. 1998) is marked with a black bar. The three possible transmembrane helices that were predicted for QcrC from C. glutamicum are represented by double lines. The two cysteine residues that coordinate one of the iron atoms are indicated by filled squares and those that form a disulfide bridge by triangles. The two histidine residues that coordinate the second iron atom are indicated by open squares.

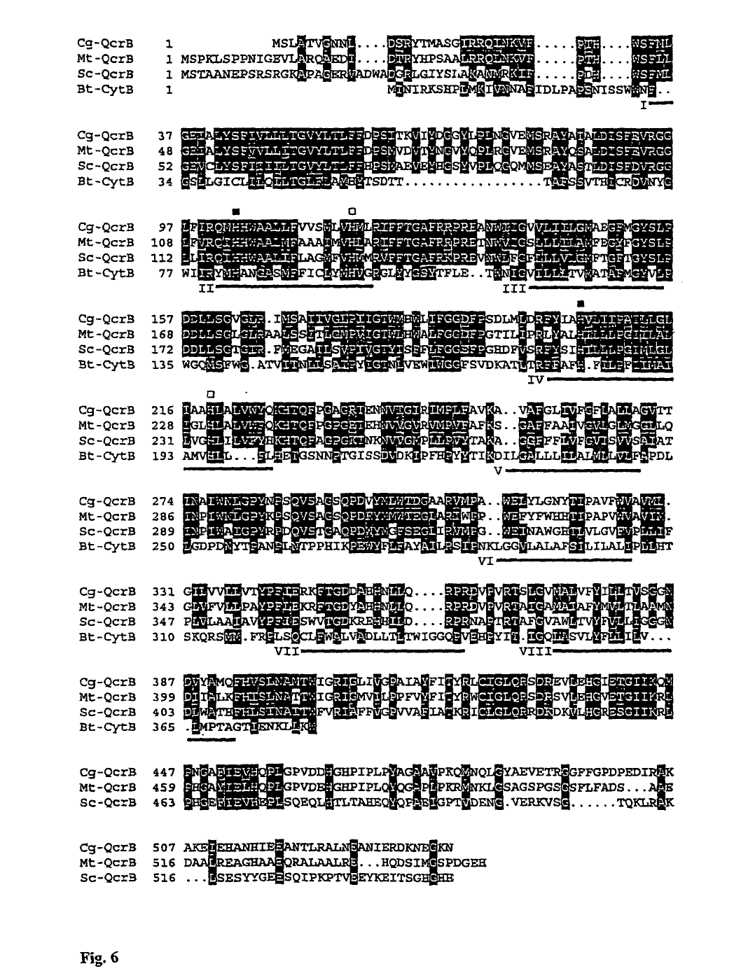

FIG. 6 is a sequence comparison of the cytochrome b subunit of the bc₁ complex from C. glutamicum (this invention), M. tuberculosis (Cole et al. 1998), S. coelicolor and cow (Anderson et al. 1982). Amino acids that are identical in at least three of the four sequences were marked with a black background, conserved amino acid exchanges were marked with a gray background. The transmembrane helices I-VIII that were identified in the crystal structure of cytochrome b from cow (Xia et al. 1997; Zhang et al 1998; Iwata et al. 1998) are marked by black bars. The histidine residues that serve as ligands for the heme-iron atoms with low (b_{L}) and high (b_{H}) potential are indicated by filled and open squares.

FIG. 7 shows a Southern blot analysis of the chromosomal DNA of wild type C. glutamicum (Track 1) and the mutant 13032ΔctaD (Track 2). Genomic DNA cut with SalI and hybridized with a DIG-labeled 1.0 kb SalI insert of the pK19ms-ΔctaD as probe.

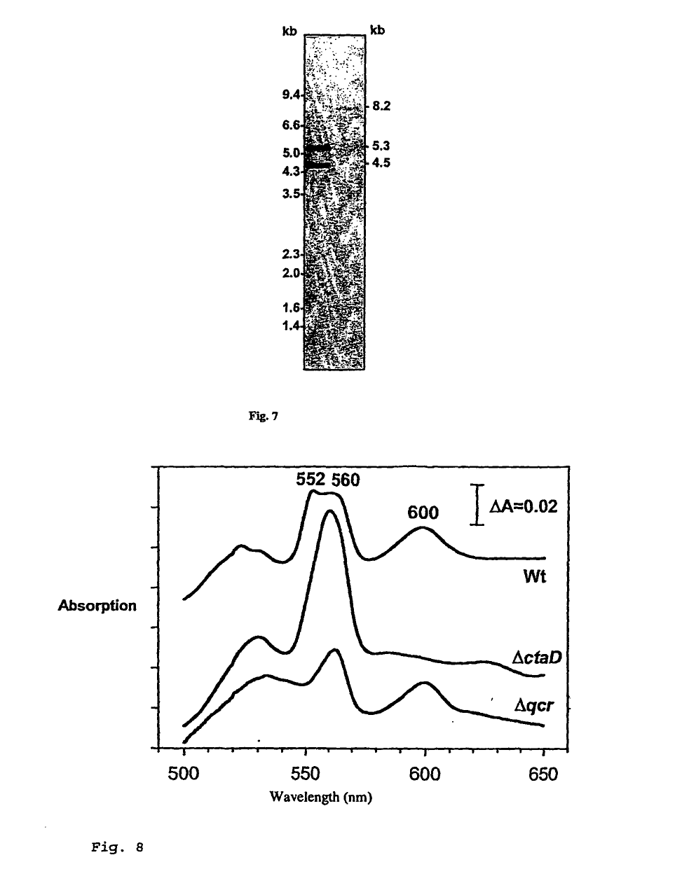

FIG. 8 shows redox difference spectra (dithionite-reduced minus ferricyanide-oxidized) of membranes (30 mg protein/mL) that were isolated from C. glutamicum ATCC13032 (Wt), ATCC13032ΔctaD (ΔctaD) and ATCC13032Δqcr (Δqcr). The cells were cultivated aerobically in BHI medium containing 2% (w/v) glucose.

FIG. 9 shows growth in CGXII minimal medium containing 4% (w/v) glucose for C. glutamicum ATCC13032 with pWKO (circles), ATCC13032 with pWK0-ctaD (filled circles), ATCC13032ΔctaD with pWKO (open triangles), and ATCC13032ΔctaD with pWK0-ctaD (filled triangles).

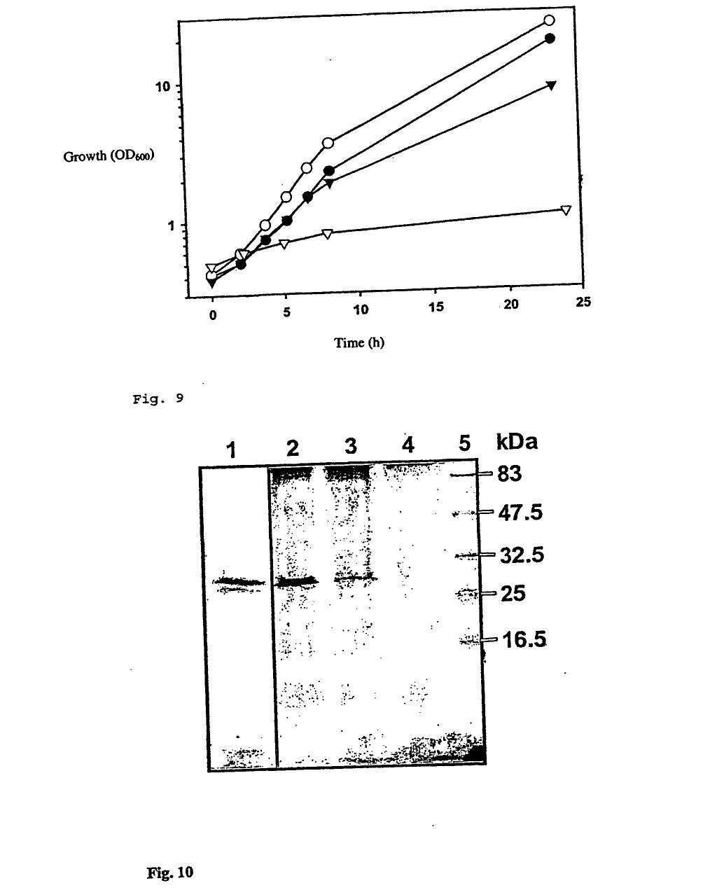

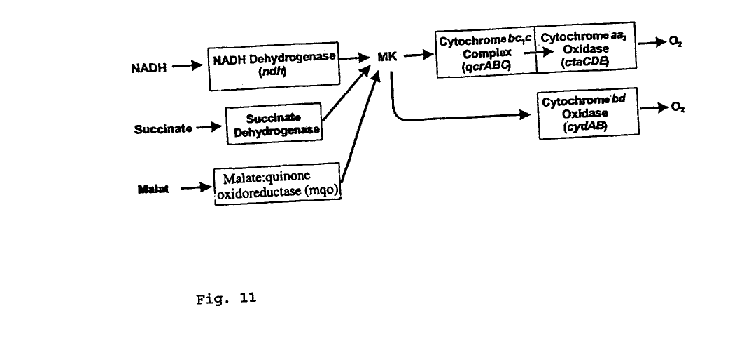

FIG. 10: C. glutamicum proteins were separated by SDS polyacrylamide gel electrophoreses and then the proteins were visualized with a covalently bonded heme group, i.e., the c type cytochrome, by staining with tetramethylbenzidine and H₂O₂ (Thomas et al. 1980). The position of cytochrome c₁ is indicated. Track 1, total cell extract (80 μg protein); Track 2, membranes (50 μg protein) of strain 13032 with plasmid pWKO, Track 3, membranes (50 μg protein) of strain 13032ΔctaD with plasmid pWKO; Track 4, membranes (50 μg protein) of strain 13032Δqcr with plasmid pJC1; Track 5, pre-stained protein standard (New England Biolabs).

FIG. 11 is a model of the branched respiratory chain of C. glutamicum.

The methods that were used are described below.

1. Cultivation of Microorganisms

Tables 1 and 2 list organism strains and plasmids that were used.

Corynebacterium glutamicum was cultivated at 30° C. either in Luria Bertani (LB) medium (Sambrook et al 1989) or in brain heart infusion (BHI) medium (Difco Laboratories, Detroit, USA) with 2% (w/v) glucose or in CGXII minimal medium with 4% glucose as carbon and energy source (Keilhauer et al. 1993). If necessary, kanamycin (25 μg/mL) was added.

Escherichia coli (E. coli) was cultivated at 37° C. in LB medium. Optionally, kanamycin (50 μg/mL) or carbenicillin (100 μg/mL) was added.

2. DNA Isolation and Transformation

Chromosomal DNA was isolated from C. glutamicum as described in Eikmanns et al (1994). Plasmids from E. coli were isolated either with the QIAprep Spin Miniprep Kit from Quiagen or with the Plasmid Maxi Kit from Quiagen. E. coli was transformed with CaCl₂ by the method of Cohen et al. (1972) or by means of electroporation following Dower et al. (1988).

C. glutamicum was transformed by electroporation and is given in van der Rest et al. (1999).

3. DNA Sequence Analysis

The DNA sequence analysis was carried out by means of the dideoxynucleotide chain termination method (Sanger et al. 1977) using a thermosequence-fluorescence sequencing kit (Amersham Pharmacia Biotech) and an automated DNA sequencer (LI-COR DNASequencer 4200, MWG-Biotech). Alternatively, the DNA was sequenced by MWG Biotech. The sequence comparisons were shaded with the help of Boxshade software. The transmembrane helices were predicted with the TopPred II software (Claros and von Heinjne 1994).

4. DNA Modifications

Restriction enzymes, T4 DNA ligase, Klenow polymerase and calf intestine alkaline phosphatase were obtained either from Roche Diagnostics or New England Biolabs.

For the Southern hybridization (Southern 1975) 1 to 5 μg genomic DNA or 1 ng plasmid DNA was completely digested with suitable restriction enzymes, fractionated on a 1% agarose gel and then transferred to a nylon membrane by means of a vacuum supported diffusion. The labeling of the samples with digoxigenin, hybridization, the washing step and detection were carried out with the DIG Chem-Link labeling and detection kit according to the directions of the manufacturer Roche Diagnostics. The DIG labeled DNA molecular weight markers II and III (Roche Diagnostics) were used for size labeling.

For colony hybridization the colonies were transferred to a nylon membrane (Hybond-N+, Amersham Pharmacia Biotech). The cell lyses and fixing of the DNA on the membrane was carried out by autoclaving (3 min at 105° C.) the filter (Kullik and Giachino 1997). After removal of cell fragments by washing with 2×SSC (300 mM, NaCl, 30 mM N₃ citrate, pH 7) the membranes were used directly for prehybridization.

The Taq-DNA polymerase was used for routine PCR. To obtain PCR products with high exactness the Expand High Fidelity PCR System (Roche Diagnostics) was used. For standard reactions a 100 μL solution containing the following components was used: 0.5 μM of the two primers that were used, 1 μg matrix DNA, 200 μM each dATP, dGTP, dCTP and dTTP, as well as 2.5 U Taq DNA polymerase. For a PCR with degenerated primers the primer concentration was increased to 4 μM. The reactions were carried out in a Primus 25 Thermocycler (MWG Biotech). For screening of E. coli transformants for the presence of recombinant plasmids individual colonies were re-suspended in 5 μL water and then used as matrices in a PCR batch with a total of 20 μL volume. To lyse the cells and release the plasmid DNA the batch was incubated for 10 min at 95° C. before the start of the actual PCR.

5. Separation of ctaD and qcrCAB Deletion Mutants

An “in-frame” ctaD deletion mutant of C. glutamicum was prepared with the help of the “crossover” PCR following Link et al. (1997) and using the suicide vector pK19mobsacB, which cannot be replicated in C. glutamicum. First two PCR products were generated, of which one included the 5′-flanking region of ctaD including the first six ctaK codons (primer pair ΔctaD-1/ΔctaD-2) and the other included the 3′-flanking region of ctaD including the last 16 ctaD codons (primer pair ΔctaD-3/ΔctaD-2). The primers ΔctaD-1 and ΔctaD-4 contain an SalI restriction cut site at the 5′ end, of the primers ΔctaD-2 and ΔctaD-3 contain at the 5′ end 21 base pairs (bp) including “tag” sequences that were complementary to each other (see Table 2).

In a second step the two resulting PCR products (543 bp and 534 bp) are purified with a PCR purification kit. Then they were mixed and used as matrix, template for the crossover PCR with the primers ΔctaD-1 and ΔctaD-4. The two matrix fragments together can form a double strand over the 21 base pair complementary “tag” sequence. The resulting fusion product of 1056 base pairs was treated with SalI, purified and cloned in the pK19mobsacB vector that had been digested with SalI and treated with alkaline phosphatase. The plasmid pK19ms-ΔctaD resulted from this. C. glutamicum was transformed with this plasmid by means of electroporation and plated out onto LBHIS agar containing kanamycin (van der Rest et al 1999). Since pK19ms=ΔctaD cannot be replicated in C. glutamicum, kanamycin-resistant clones integrated the plasmid into their chromosome through homologous recombination via one of the ctaD flanking regions. To select a second recombination product, a kanamycin resistant clone was cultivated for 24 h in BHI medium with 2% (w/v) glucose and 5 mM sodium azide and then plated out onto LBHIS agar plates with 10% (w/v) sucrose. In the presence of levansucrase, the product of the sacB gene, which is contained in pK19ms-ΔctaD plasmid, a concentration of 10% sucrose has a lethal effect on C. glutamicum. Therefore, in sucrose-resistant clones the vector fragment (pK19mobsacB) of the pK19ms-ΔctaD plasmid should have been cut out by a second homologous recombination product, so that either the wide type situation is re-established or, as desired, the deletion of the ctaD gene is achieved. Clones that were kanamycin sensitive and sucrose resistant were initially analyzed by means of PCR. For this, genomic DNA was isolated and a PCR was carried out with the primers ΔctaD-1 and ΔctaD-4. Clones that produced the desired PCR products in this PCR were then again controlled by means of Southern blot. For this, the chromosomal DNA was digested with SalI and a DIG labeled 1.0 kb SalI fragment from the plasmid pK19ms-ΔctaD was used as probe.

A qcrCAB deletion mutant of C. glutamicum was constructed by the same principle that was described above for the ctaD. The 5′-flanking region of qcrC was amplified with the primers Δqcr-1 and Δqcr-2, and the 3-flanking region of qcrB was amplified with the primers Δqcr-3 and Δqcr-4. The resulting PCR fragments were used for a crossover PCR with the primers Dqcr-1 and Dqcr-4, forming a 1062 bp PCR product. After digestion of this fragment of SalI and treatment with alkaline phosphatase it was cloned into the vector pK19mobsacB, from which the plasmid pK19ms-Δqcr resulted. Then the strain 13032Δqcr was constructed with the help of this plasmid. The successful deletion of the qcrCAB genes were controlled first by PCR with genomic DNA of the strain and the primers Δqcr-1 and Δqcr-4 and then by Southern blot analysis with SalI digested chromosomal DNA and the DIG-labeled 1.0 kb SalI fragment for pK19ms-Δqcr as probe. In the wild type DNA the hybridizing fragment of 12 kb was detected, while in the Δqcr mutant a hybridizing of 8 kb was found. With that the deletion of the 3.7 kb qcrCAB fragment from the chromosome was confirmed.

6. Membrane Isolation, Heme Staining and Different Spectroscopy

10 g cell mass (wet weight) as suspended in 15 mL 100 mM tris/HCl, pH 7.5 with 1 mM PMSF. Cell disruption was carried out, in which the suspension was passed 5 times at 207 MPa through a French press (SLM Aminco). Cell fragments were removed by centrifuging at 8000 g for 15 min. The supernatant (cell-free extract) was again centrifuged for 90 min at 150,000 g. The residue with the cytoplasmid membrane was re-suspended in 10 mM tris/HCl at pH 7.5 (50-80 mg protein/mL) and stored for further analyses. SDS-PAGE was carried out in accordance with Laemmli (1970) except that the samples were first incubated for 30 min at 40° C. Staining of proteins with covalently bonded heme groups was carried out with tetramethylbenzidine in accordance with Thomas et al. (1980). Dithionite-reduced minus ferricyanide-oxidized different spectra were obtained at room temperature with a Jasco V560 spectrophotometer, which was equipped with a silicon photodiode detector for turbid samples (Castiglioni et al. 1997). Here a cuvette with a 5 mm wide detection window was used. The protein concentration was carried out with the bicinchoninic acid (BCA) protein analysis (Smith et al. 1985) and bovine serum of albumin as standard.

Embodiment Examples

A) Identification of the ctaD Gene that Codes for Subunit I of a Terminal Oxidase of the Heme-Copper Family

Reduced minus oxidized different spectra and CO reduced minus reduced different spectra point to the existence of a cytochrome c oxidase of aa₃ type in C. glutamicum (Trutko et al. 1982). The fact that the primary sequence of subunit I of the heme-copper oxidases, to which cytochrome aa₃ belongs, is highly conserved was employed in this invention to clone the pertinent genes. Based on earlier sequence comparisons (Bott et al. 1990; Bott et al. 1992) three regions were chosen for the derivation of degenerated primers (Table 1): WFFGHPE, which contains one of the Cu_{B} histidine ligands (primer ctaD-for1), VWAHHM, which contains the two Cu_{B} histidine ligands (primer ctaD-for2, ctaD-rev2), and AHFHYV, which contains a heme a and a heme a₃ histidine ligand (primer ctaD-rev1). Using chromosomal C. glutamicum DNA as matrix PCR products of the expected sizes were obtained with all primer combinations: a 0.43 kb DNA fragment with ctaD-for1/ctaD-rev1, a 0.17 kb product with ctaD-for1/ctaD-rev2 and a 0.28 kb produce with ctaD-for2/ctaD-rev1. The 0.17 kb and the 0.28 kb PCR products were also obtained when the purified 0.43 kb PCR product was used as matrix instead of the chromosomal DNA. The final corroboration that the PCR products are a part of the ctaD gene that codes for subunit 1 of heme-copper oxidase was obtained through the sequential analysis of the 0.43 kb fragment. This analysis was carried out after cloning the 0.43 kb fragment into the vector pCR2.1TOPO, resulting in the plasmid pCR.2-ctaD. A Southern blot analysis with the DIG-labeled 0.43 kb fragment as probe showed that the ctaD gene of C. glutamicum was localized on a chromosomal BamHI fragment of 3.5 kb and EcoRI fragment of 9.0 kb, a HindIII fragment of 2.8 kb, an SalI fragment of 5.2 kb and a XhoI fragment of 8.0 kb.

A cosmid gene bank of C. glutamicum (Böermann et al. 1992) was hybridized with the DIG-labeled 0.43 kb ctaD fragment in order to clone the complete ctaD gene sequence. Cosmid DNA of one of the positive clones was digested with EcoRI and the resulting 9.0 kb fragment was subcloned in pUC18, producing pUC18-CE. A restriction analysis of pUC18-CE proved that it contained the ctaD gene. A 4.0 kb EcoRI/NcoI fragment and the 2.4 kb NcoI/BamHI fragment of pUC18-CE were cloned in pUC-BM20 for sequence analysis and in this way the plasmids pBM20-CEN and pBM20-CNB were obtained. Both strands of a 3000 bp region that included the ctaD gene were sequenced by means of a primer walking strategy. The sequence analysis revealed three genes (FIG. 1A): position 1 to 608 was identical with the 3′ end of the nrdF gene, which codes for the β subunit of the ribonucleotide reductase (Oelmann and Auling 1999). The ctaD gene that codes for the subunit I of a heme-copper oxidase begins 521 bp beyond the nrdF stop codon at position 1120 with ATG and ends at position 2883 with TAA.

B) Characterization of the Primary Structure of CtaD

The protein derives from the ctaD gene consists of 584 amino acids with a molecular weight of 65102 Da. It is discernable from the sequence comparison (see FIG. 2) that the CtaD protein is the subunit I of a terminal oxidase from the heme-copper family. The protein showed sequence identity of 72%, 65% and 42% with the corresponding proteins of M. tuberculosis, Streptomyces coelicolor and Paracoccus denitrificans. All ligands of heme a (His-97, His-400), heme a₃ (His-398) and Cu_{B} (His-265, His-314, His-315) were present, as well as many other functionally important residues (Review Garcia-Horsman et al. 1994, Ferguson-Miller and Babcock, 1996). A hydrophobicity analysis predicted 12 transmembrane helices, whose arrangement corresponded with the 12 transmembrane helices from P. denitrificans CtaD (FIG. 2), which were identified by means of the x-ray structure of this protein (Iwata et al. 1995).

C) Identification and Sequence of the ctaE-qcrACG Genes that Code for Subunit III of a Terminal Oxidase of the Heme-Copper Family and a Cytochrome bc₁ Complex

The presence of a peak at 550 nm in the redox difference spectra of C. glutamicum membranes, which is for c type cytochromes, pointed to the presence of a menaquinone cytochrome c oxidoreductase in this organism. As with the identification of the ctaD gene a PCR was carried out with degenerated primers from conserved regions in order to identify the corresponding genes. For this, three regions of the QcrC and QcrA proteins from M. tuberculosis were chosen (Table 1): SCVSCH, which contains the covalent binding site for heme c₁ (primer qcrC-for), CASCHN, which contains a second covalent binding site (primer qcrC-rev), and CPCHQS, which contains a cysteine and histine ligand of the Rieske iron-sulfur protein (primer qcraA-rev). Neither of the two primer combinations (qcrC-for/qcrC-rev, qcrC-for/qcrA-rev) produced PCR products of the expected size of chromosomal DNA from C. glutamicum as matrix. Since in M. tuberculosis the ctaE gene that codes for subunit III of a heme-copper oxidase is localized this qcrC gene (Cole et al. 1998), two conserved regions of the CtaE protein were chosen for derivation of additional primers: TGFHGLHV (primer ctaE-for1) and YYWHFVD (primer ctaE-for2 and ctaE/rev1). PCR products with sizes of 2.2 kb, 0.84 kb and 0.14 kb were obtained with the primer combinations ctaE-for1/qcrA-rev, ctaE-for1/qcrC-rev and ctaE-for1/ctaE-rev1. The primer combinations ctaE-for2/qcrA-rev and ctaE-for2/qcrC-rev produced PCR products with sizes of 2.1 kb and 0.72 kb. When the purified 2.2 kb PCR fragment was used as matrix it was possible to obtain with the primer combination ctaE-for1/ctaE-rev1 a 0.14 kb PCR product, from which it could be concluded that the 2.2 kb PCR product contained the expected DNA region. A final confirmation that the 2.2 fragment contains the 3′ end of the ctaE gene, the complete qcrC gene and the 5′ end of the qcrA gene was obtained by partial sequence analysis of the fragment, after which it was cloned in vector pCR2.1-TOPO, which lead to the plasmid pCR2.1-cta/qcr. After supplementing the obtained sequence data with those from the C. glutamicum genome sequencing project of Degussa AG it was possible to obtain the sequence of the 3′ end of the ctaE gene and the complete sequence of the acrCAB genes. However, since the 5′ end of the ctaE gene was still lacking, a hybridization of the cosmid gene bank of C. glutamicum (Börmann et al. 1992) was carried out with the DIG-labeled 2.2 kb PCR product. In this way a cosmid was isolated that contained the missing 5′ end of the ctaE gene and then the corresponding region could be sequenced.

FIG. 1B presents the physical map of the 5709 bp region that contains the genes ctaE and qcrCAB. The coding regions are arranged at the following sites: ctaE (subunit III of the heme-copper oxidase) 570-1187; qcrC (cytochrome c₁) 1209-2149; qcrA (“Rieske” iron-sulfur protein) 2146-3372, qcrB (cytochrome b) 3369-4988.

D) Analysis of Primary Structure of the CtaE, QcrC, QcrA and QcrB Proteins

The ctaE gene codes for a protein with 205 amino acid residues and a molecular weight of 22442 Da. The amino acid sequence showed 61%, 58% and 35% correspondence with the corresponding protein from M. tuberculosis, S. coelicolor and P. denitrificans. It can be seen in the sequence comparison (FIG. 3) that the CtaE protein of the three gram-positive bacteria lack the N-terminal region of the CtaE protein from P. denitrificans. A hydrophobicity analysis showed that the CtaE proteins of C. glutamicum, M. tuberculosis and S. coelicolor contain five probable transmembrane helices, which are represented in FIG. 3.

The qcrC gene codes for a protein with 283 amino acid residues and a molecular weight of 29863 Da. QcrC contains heme-bonding sites, which are characteristic c type cytochromes: CITCH and CASCH, which begin at position 67 and 177, respectively. This indicates that QcrC is a diheme cytochrome c. Cys-67 and Cys-70 function as ligands for covalent bonding of the first heme group and His-71 and Met-102 function as axial ligands of the corresponding heme-iron atoms. Cis-177 and Cys-180 presumably serve as ligands for covalent bonding of a second heme group and His-181 as well a Met-211 or Met-218 serve as axial ligands for the second heme-iron atom. A hydrophobicity analysis indicated that the QcrC presumably has two transmembrane helices, one in N-terminal position (position 20 to 40) and another in C-terminal position (position 261 to 281). However, the N-terminal sequence does not correspond with the typical signal sequence that is used for Sec-depend export. Although two pairs of adjacent arginine residues are present (Arg-12, Arg-13, Arg-16, Arg-17), the adjacent residues do not correspond with the twin arginine motif of proteins, which are exported via the alternative Tat pathway (Berks, 1996). Therefore it is possible that the QcrC protein is accurate in the membrane via a N-terminal and a C-terminal transmembrane helix. The QcrC protein from C. glutamicum shows the greatest correspondence with the QcrC protein from M. tuberculosis (59%) and S. coelicolor (52%) which likewise has two heme binding sites, as shown in the sequence comparison in FIG. 4.

The qcrA gene codes for a protein with 408 amino acids and a molecular weight of 45184 Da. As shown in FIG. 5, QcrA contains two conserved sequence structures, which are characteristic for 2Fe-2S Rieske iron-sulfur proteins: CTHIG and CPCH, which begin at positions 333 and 352, respectively. Cys-333 and Cys-352 coordinate one of the non-heme-iron atoms, His-335 and His-355 coordinate the second non-heme-iron atom, while Cys-338 and Cys-354 form a disulfide bridge (Iwata et al. 1996). Data bank investigations showed the greatest correspondence with QcrA from M. tuberculosis (52% identity), followed by QcrA from S. coelicolor (34% identity). The correspondence with the Rieske iron-sulfur protein of the bc₁ complex from bovine heart only showed a correspondence of 22%. Compared with the mitochondrial protein, which has a single transmembrane helix at the N-terminus, the QcrA polypeptide from C. glutamicum, M. tuberculosis and S coelicolor contain an extensive N-terminus with three potential transmembrane helices.

The qcrB gene codes for a protein with 539 amino acids and a molecular weight of 59809 Da. Sequence comparisons showed a correspondence of 63%, 47% and 26% with QcrB from M. tuberculosis, QcrB from S. coelicolor and the cytochrome b subunit of the bc₁ complex from bovine heart mitochondria. FIG. 6 shows that the histidine residues that serve as ligands for the heme-iron atom with low potential (b_{L}) are localized at position 103 and 204, and those that serve as ligands for the heme-iron atom with high potential (b_{H}) are localized at positions 117 and 219. The crystal structure of the bc₁ complex from bovine heart (Xia et al. 1997; Iwata et al. 1998) showed that cytochrome b contains eight transmembrane helices (TMHs), which are designated A through H. The histidine ligands are localized in B and D. In addition, there are four other “horizontal” helices localized on the positive side of the membrane (corresponds to the periplasmatic or extracytoplasmatic space in the case of bacteria), which are designated as ab, cd1, cd2, and ef. Hydrophobicity analyses showed nine transmembrane helices for the QcrB sequences for C. glutamicum, M. tuberculosis and S. coelicolor. The fourth of these helices corresponds with the peripheral cd1 and cd2 helices. The two largest differences between the QcrB sequence and the sequence of cytochrome b from cow are evident from FIG. 6: and extension of 17 amino acids in the extracytoplasmatic part, which bind the helices A and B, and large domain of about 120 amino acids at the C-terminus of QcrB, which is not present cytochrome b from bovine heart. This additional C-terminal domain is detectable in all currently known cytochrome b sequences from Corynebacterium-, Mycobacterium-, and Streptomyces species.

E) Construction and Phenotype of a C. glutamicum ctaD Deletion Mutant

A deletion mutant was constructed in order to show that the ctaD gene product is a subunit of the cytochrome aa₃ oxidase and not an additional oxidase from the heme-copper superfamily that could be present in C. glutamicum. Crossover PCR (Link et al., 1997) and the suicide vector pK19mobsacB (Schafer et al. 1994) was used for this purpose. The codons 7 through 569 of ctaD were exchanged for a 21 bp sequence “tag” in the resulting strain 13032ΔctaD (see Description of Methods). To verify the genomic structure of the mutant, the chromosomal DNA of the wide type and the mutant was cut with SalI and analyzed by Southern blot, where a DIG labeled 1.0 kb SalI fragment from the plasmid pK10ms-ΔctaD was used as probe. Two hybridizing fragments of 5.2 kb and 4.5 kb in size could be detected in the wild type DNA, whereas in the mutant only a single 8.1 kb fragment was shown (FIG. 7). This confirms the expectation that the ctaD internal SalI cut site is absent in the deletion mutant.

Redox difference spectra (dithionite reduced minus ferricyanide oxidized) of membranes that were isolated from the wide type and the ctaD mutant showed that the 600 nm peak that indicates the presence of heme a, was detectable only in the wild type and not in the mutant (FIG. 8). This result confirms the assumption that the ctaD product is identical with subunit I of cytochrome aa₃ oxidase from C. glutamicum. Further evidence for this conclusion was achieved through transformation of the 13032ΔctaD mutant with the plasmid pWK0-ctaD, which contained the ctaD gene under control of the pertinent promoter. The different spectrum of the complimentary strain again showed the 600 nm peak.

While the growth of the ΔctaD mutant in BHI medium with 2% (w/v) glucose was nearly unaffected in comparison with the wild type, in CGXII minimal medium with 4% (w/v) glucose almost no growth was observed. This clear defect could be partially reversed by transformation of the ΔctaD mutant with the plasmid pWKO-ctaD with an intact ctaD gene.

F) Detection of an Individual c Type Cytochrome in Membranes of C. glutamicum

The identification of a cytochrome in C. glutamicum with two CXXCH motifs for covalent heme-bond indicates that this protein is a diheme cytochrome c. One possible function of the second heme group could be electron transfer to cytochrome aa₃ oxidase, where a separate cytochrome c which would normally participate in this process, was replaced. In order to determine the number and size of the c type cytochromes in C. glutamicum, a membrane fraction and a soluble fraction of wild type cells, which were cultivated aerobically in complete medium was prepared. Aliquots of both fractions were separated by SDS-PAGE and proteins with covalently heme groups were stained (Thomas et al. 1980). As can be seen in FIG. 10, with this method an individual c cytochrome, which could be identical to cytochrome c₁ (molecular weight of the Apo form 30 kDa) because of the apparent molecular weight of about 31 kDa, was detected in the membrane fraction. The stained band with the apparent molecular weight of about 100 kDa possibly represents the un-disassociated form of the bc₁ complex and not another c type cytochrome. After staining no protein with covalently bonded heme could be detected in the soluble fraction of C. glutamicum.

G) Construction and Phenotype of a C. glutamicum qcrCAB Deletion Mutant

To prove that the c type cytochrome with an apparent molecular weight of 32 kDa indeed represents cytochrome c₁, a C. glutamicum qcrCAB deletion mutant was constructed and checked by Southern blot analysis. A heme staining of the membrane fraction of strain 13032Δqr by SDS-PAGE showed that the 31 kDa heme protein was lacking and therefore as expected [it] is identical to cytochrome c₁. Further support for this came from the reduced minus oxidized different spectra of membranes of strain 13032Δqcr, in which the 550 nm peak for c type cytochromes was absent. Similar to the case of strain 13032ΔctaD, the growth of strain 13032Δqcr in CGXII minimal medium with 4% (w/v) glucose was adversely affected significantly and this effect could be reversed by complementation with the qcrCAB expression plasmid bJC1-bc_{His}.

Consequences for the Respiratory Chain:

As already described above, the qcrCAB genes code for the cytochrome bc₁ complex and the gene ctaD and ctaE code for the subunits I and III of cytochrome aa₃ oxidase in C. glutamicum. With the exception of CtaD, all the proteins that derived from these genes showed clear differences with the well-studied corresponding proteins from other bacteria and mitochondria. Subunit III of cytochrome aa₃ oxidase (CtaE) does not contain the N-terminal part of the corresponding protein from P. denitrificans, due to which a protein that contains only five, compared to the usual seven, transmembrane helices is formed (FIG. 3). The Rieske iron-sulfur protein (QcrA) is clearly larger than the corresponding protein for many other bacteria and mitochondria, namely because of an N-terminal extension of about 200 amino acids. Hydrophobicity analyses point to the existence of three transmembrane helices in this part (FIG. 5), while the “classic” Rieske iron-sulfur protein in its end form has only a single N-terminal transmembrane helix.

The cytochrome b subunit (QcrB) differs from the corresponding mitochondrial protein by a large C-terminal extension of about 130 amino acids. Potential transmembrane helices were not found within the last 120 amino acid residues, which indicates that this extension represents a soluble domain, which should be localized in the cytoplasm because of the structure of the mitochondrial cytochrome b. The function of this additional domain of cytochrome b from C. glutamicum is not yet clear.

The interesting factor within the cytochrome bc₁ complex of C. glutamicum was found in cytochrome c₁: in contrast to the traditional bc₁ complex to CXXCH motifs were found for covalent bonding of heme B in the primary structure (FIG. 4). This indicates with a great probability that the protein is a diheme and not a monoheme c type cytochrome. Since cytochrome c₁ probably is the only c type cytochrome in aerobically grown cells of C. glutamicum (FIG. 10), it is obvious to assume that the second heme group in cytochrome c₁ takes on the function of a second c type cytochrome and the cytochrome aa₃ oxidase takes over electron transfer from cytochrome c₁ to the Cu_{A} center of subunit II. If this assumption is correct, the cytochrome bc₁ complex from C. glutamicum (and possibly also from M. tuberculosis and S. coelicolor) functions as menaquinone cytochrome aa₃ oxidoreductase. This function would necessitate close contact between cytochrome c₁ and the Cu_{A} electron entry sites in cytochrome aa₃. It is possible that both complexes form a supercomplex, as was already described for P. denitrificans (Berry and Trumpower, 1985), the thermophilic bacterium PS3 (Sone et al. 1987) and mitochondria (Schagger and Pfeiffer 2000; Cruciat et al. 2000). If a bc₁-aa₃ supercomplex is not formed the frequency of the collision of the two complexes in the membrane must be so rapid that a sufficiently high electron transfer rate is achieved. A cytochrome bc₁ complex with a diheme cytochrome c₁ is also assumed in the case of Heliobacillus mobilis, since the petX gene product contains two CXXCH patterns for covalent heme bonding (Xiong et al. 1998).

In many bacteria, for example P. denitrificans (Steinrücke et al. 1991) or Bradyrhizobium japonicum (Bott et al. 1990, 1992), the deletion or inactivation of the cytochrome aa₃ oxidase genes does not have a significant effect on growth behavior, since the function is taken over by one of the other alternative oxidases. In comparison a ctaD deletion mutant of the C. glutamicum strain 13032 grew well in BHI medium, but extremely poorly in glucose minimal medium (FIG. 9). Although the reason for this difference is still not known, it does show the importance of the bc₁-aa₃ pathway of the respiratory chain for growth in minimal medium.

FIG. 11 shows the branched respiratory chain of C. glutamicum. The NADH formed by oxidation of carbon sources is oxidized via the product of the ndh gene (access number AJ238250), which codes for a membrane-bound non-proton pumping NADH dehydrogenase, which is called NDH-2 (Molenaar et al. 2000). Whether C. glutamicum also has a proton-pumping NADH dehydrogenase (NDH-1), as is suggested by the existence of the gene nuoU, nuoV and nuoW, is still not unambiguously certain. Besides the NADH dehydrogenase there are at least two other enzymes that transport electrons to menaquinone during aerobic growth: succinate-dehydrogenase and malate:quinone oxidoreductase (Molenaar et al. 1998). The menaquinone reduced by these enzymes is either oxidized by the cytochrome bc₁ complex, which presumably transports electrons directly to the terminal cytochrome aa₃ oxidase, or alternatively is oxidized by the cytochrome bd terminal oxidase. These two pathways differ with regard to bioenergetic efficiency. In correspondence with generally recognized values the H⁺/2e⁻ ratio for the bc₁-aa₃ pathway should be six (Nicholls and Ferguson 1992), whereas it should be only two for the cytochrome bd terminated pathway (Miller and Gennis 1985; Puustinen et al. 1991). Based on these assumptions the P/O ratio of the bc₁/aa₃ pathway is greater than that of the cytochrome bd pathway by a factor of 3.

In evaluating the efficiency of the respiratory chain of C. glutamicum one must include the results of Schirawski and Unden (1998) which indicate that the electron transport of succinate (E′₀=+30 mV) to menaquinone (E′₀=−80 mV), catalyzed by succinate dehydrogenase, implies a reversed electron transport through the cytoplasm membrane, which is driven by the electrochemical proton potential. In other words, a part of the electrochemical proton potential that is formed by respiration would have to be used for the oxidation of succinate in the citrate cycle instead of for ATP synthesis by the F₀F₁-ATP synthase. If the cytochrome bd oxidase is the only coupling site in the respiratory chain, as is presumably the case in the ΔctaD mutant, it would be possible that C. glutamicum obtains ATP exclusively via substrate stage phosphorylation and not via oxidative phosphorylation.

TABLE 1
Bacterial strains and plasmids that were used

| Strain/plasmid | Relevant characteristics | Source or reference |
|---|---|---|
| Strain | | |
| Escherichia coli | | |
| DH5α | F Φ80dlacZΔM15 Δ(lacZYA-argF) U169 endA1 | Gibco BRL |
| | recA1 hsdR17 deoR thi-1 supE44 λ⁻gyrA96 relA1 | |
| TOP10 | F Φ80lacZΔM15 ΔlacX74-mcrA Δ (mrr-hsdRMS- | Invitrogen |
| | mcrBC) deoR recA1 araD139 Δ(ara-leu) 7697 galU | |
| | galK rpsL (Str^{R}) endA1 nupG | |
| Corynebacterium glutamicum | | |
| ATCC13032 | Wild type | Abe et al. 1967 |
| 13032ΔctaD | Labeled in-frame ctaD deletion mutant of | This work |
| | ATCC13032 | |
| 13032Δqcr | Labeled qcrCAB deletion mutant of ATCC13032 | This work |
| Plasmid | | |
| pCR2.1-TOPO | AP^{R} Km^{R}; cloning vector for PCR products with | Invitrogen |
| | 3′-A overhand | |
| pUC18 | AP^{R}; cloning vector | Yanish-Perron et al. 1985 |
| pUC-BM20 | AP^{R}; cloning vector | Boehringer Mannheim |
| pK19mobsacB | Km^{R}; mobile E. coli for construction of | Schäfer et al. 1994 |
| | C. glutamicum insertion and deletion mutants | |
| pWKO | Km^{R}; mobile low-copy number E. coli- | Reinscheid et al. 1994 |
| | C. glutamicum shuttle vector | |
| pCR2.1-ctaD | AP^{R} Km^{R}; PCR2.1-TOPO derived from 0.43 kb ctaD | This invention |
| | PCR fragment obtained with the primers ctaD-for1 | |
| | and ctaD-rev1 | |
| pUC18-CE | AP^{R}; pUC18 derived from ctaD contains 9 kb EcoRI | This invention |
| | fragment from cosmid pHC70-ctaD | |
| pBM20-CEN | Ap^{R}; pUC-14-BM20 derived from 4.0 kb EcoRI- | This invention |
| | NcoI fragment from pUC18-CE | |
| pBM20-CNB | AP^{R}; pUC-14-BM20 derived from 2.4 kb NcoI- | This invention |
| | BamHI fragment from pUC18-CE | |
| pCR2.1-cta/qcr | AP^{R}; pCR2.1-TOPO derived with the 2.2 kb PCR | This invention |
| | fragment obtained with the primer ctaE-for1 and | |
| | qcrA-rev; contains the 3′ end of ctaE, the complete | |
| | qcrC and the 5′ end of qcrA | |
| pK19ms-ΔctaD | Kan^{R}; pK19mobsacB derivative, which carries a | This invention |
| | 1056 bp crossover PCR product in the SalI cut site, | |
| | which includes the 5′- and 3′-flanking region of the | |
| | C. glutamicum ctaD gene | |
| pWKO-ctaD | Kan^{R}; pWK0 derived contains a 2.53 kb DraI-NaeI | This invention |
| | fragment with the C. glutamicum ctaD genes cloned | |
| | in the SalI site by a Klenow fill-in reaction | |
| pK19ms-Δqcr | Kan^{R}; pK19mobsacB derivative, which carries a | This invention |
| | 1062 pb crossover PCR product in the SalI cut site, | |
| | which includes the 5′-flanking region of qcrC and the | |
| | 3′-flanking region of qcrB | |
| pJC1 | Kan^{R}; E. coli-C. glutamicum “Shuttle”-Vektor | Cremer et al. 1990 |
| pJC1-bc_{His} | Kan^{R}; pJC1 derivative with a 5.0 kb PCR fragment, | This invention |
| | that was obtained with the primers bc-h6-for and bc- | |
| | h6-rev and contains the C. glutamicum ctaE-qcrCAB | |
| | genes. The fragment was cloned in SalI-XbaI | |
| | digested pJC1 vector. The qcrB gene carries 6 | |
| | additional histidine codons at 3′ end | |

TABLE 2

Oligonucleotides that were used

| | |
|---|---|
| Name | Sequence |

| | |
|---|---|
| ctaD-for1 | 5′-TTGTT (CT) TT (CT) GGICA (CT) CCIGA-3′ [16-fold] |
| ctaD-for2 | 5′-GTITGGGCICA (CT) CA (CT) ATGTT (CT)-3′ [8-fold] |
| ctaD-rev2 | 5′-AACAT (AG) TG (AG) TGIGCCCAIAC-3′ [4-fold] |
| ctaD-rev1 | 5′-AC (AG) TA (AG) TG (AG) AA (AG) TGIGC-3′ [16-fold] |
| ctaE-for1 | 5′-ACIGGITT (CT) CA (CT) GGI (CT) TICA (CT) GT-3′ [16-fold] |
| ctaE-for2 | 5′-TA (CT) TA (CT) TGGCA (CT) TT (CT) GTIGA (CT)-3′ [32-fold] |
| ctaE-rev1 | 5′-(AG) TCIAC (AG) AA (AG) TGCCA (AG) TA (AG) TA-3′ [32-fold] |
| qcrC-rev | 5′-(AG) TT (AG) TG (AG) CAI (GC) (AT) IGC (AG) CA-3′ [64-fold] |
| qcrA-rev | 5′-(GC) (AT) (CT) TG (AG) TG (AG) CAIGG (AG) CA-3′ [64-fold] |
| ΔctaD-1 | 5′-ACTGTCGACGGCTGTAGTTAACTGCAACCG-3′ |
| ΔctaD-2 | 5′-CCCATCCACTAAACTTAAACAAGGCGCCACAGCGGTCATAGG-3′ |
| ΔctaD-3 | 5′-TGTTTAAGTTTAGTGGATGGGCCAGAATTGGGTACCGCCCCA-3′ |
| ΔctaD-4 | 5′-ACTGTCGACGGTCTCGACAGG-3′ |
| Δqcr-1 | 5′-ACTGTCGACCTCAACGTGCCCTACGCAC |
| Δqcr-2 | 5′-CCCATCCACTAAACTTAAACATGGGGTCTGCGGGTTGGTTCC |
| Δqcr-3 | 5′-TGTTTAAGTTTAGTGGATGGGGAGGCAAACATTGAGCGTGACAA |
| Δqcr-4 | 5′-TGAGTCGACCTGCAATTTCAGGAAACTTCC |
| bc-h6-for | 5′-ACTTCTAGATAGGGTTGACATTTTGTC |
| bc-h6-rev | 5′-AGTGTCGACCTAATGGTGATGGTGATGGTGAGCTGCGTTCTTGCCCTCATTCTTGTC |

Footnote:
“I” indicates inosine.
Where relevant the variability of the oligonucleotides is indicated in parenthesis.
The SalI and XbaI restriction cut sites in the oligonucleotides ΔctaD-1, ΔctaD-4, Δqcr-1, Δqcr-4, bc-h6-for, bc-h6-rev and the complementary 21 bp tag sequences in ΔctaD-2 and ΔctaD-3 or Δqcr-2 and Δqcr-3 as well as the fixed histidine codons of bc-h6-rev are underlined.

Literature

Abe S, Takayama K, Kinoshita S (1967) Taxonomical studies on glutamic adic producing bacteria. J Gen Appl Microbiol 13:279-301

Anderson S, de Bruijn M H L, Coulson A R, Eperon I C, Sanger P. Young I G (1982) Complete sequence of bovine mitochondrial DNA. Conserved features of the mammalian mitochondrial genome. J Mol Biol 156:683-717

Arslan E, Schulz H, Zufferey R, Künzler P, Thöny-Meyer L (1998) Overproduction of the Bradyrhizobium japonicum c-type cytochrome subunits of the cbb₃ oxidase in Escherichia coli. Biochem Biophys Research Commun 251:744-747

Berks B S (1996) A common export pathway for proteins binding complex redox factors? Mol Microbiol 22:393-404

Berry E A, Trumpower B L (1985) Isolation of ubiquinol oxidase from Paracoccus denitrificans and resolution into cytochrome bc₁ and cytochrome c-aa₃ complexes. J Biol Chem 260:2458-2467

Börmann E R, Eikmanns B J, Sahm H (1992) Molecular analysis of the Corynebacterium glutamicum gdh gene encoding glutamate dehydrogenase. Mol Microbiol 6:317-26

Bott M, Bollinger M, Hennecke H (1990) Genetic analysis of the cytochrome c-aa₃ branch of the Bradyrhizobium japonicum respiratory chain. Mol Microbiol 4:2147-2157

Bott M, Preisig O, Hennecke H (1992) Genes for a second terminal oxidase in Bradyrhizobium japonicum. Arch Microbiol 158:335-343

Casiglioni E, Grilli E, Sanguinetti S (1997) A new simple an low cost scattered transmission eccessory for commercial double beam ultraviolet-visible spectrophotometers. Rev Sci Instrum 68:4288-4289

Claros M G, von Heijne G (1994) TopPred II: an improved software for membrane protein structure predictions. Comput Appl Biosci 10:685-686

Cohen S N, Chang A C Y, Hsu L (1972) Nonchromosomal antibiotic resistance in bacteria: genetic transformation of Escherichia coli by R-plasmid DNA. Proc Natl Acad Sci USA 69:2110-2114

Cole S T, Brosch R, Parkhill J. Garnier T, Churcher C, Harris D et al (1998) Deciphering the biology of Mycobacterium tuberculosis from the complete genome sequence. Nature 393:537-544

Collins M D, Pirouz T, Goodfellow M, Minnikin D E (1977) Distribution of menaquinones in actinomycetes and corynebacteria. J Gen Microbiol 100:221-230

Cremer U, Eggeling L, Sahm H (1990) Cloning the dapA dapB cluster of the lysine-secreting bacterium Corynebacterium glutamicum. Mol Gen Genet 220:478-480

Cruciat C, Brunner S, Baumann F, Neupert W, Stuart R A (2000) The cytochrome bc₁ and cytochrome c oxidase complexes associate to form a single supracomplex in yeast mitochondria. J Biol Chem 275:18093-18098

Dower W J, Miller J F, Ragdale C W (1988) High efficiency transformation of E. coli by high voltage electroporation. Nucleic Acids Res 16:6127-6145

Eggeling L, Sahm H (1999) L-Glutamate and L-lysine: traditional products with impetuous developments. Appl Microbiol Biotechnol 52:146-153

Eikmanns B J, Thum-Schmitz N, Eggeling L, Luedtke K U, Sahm H (1994) Nucleotide sequence, expression and transcriptional analysis of the Corynebacterium glutamicium gltA gene encoding citrate synthase. Microbiology 140:1817-1828

Ferguson-Miller S, Babcock G T (1996) Heme/copper terminal oxidases. Chem Rev 96:2889-2907

Garcia Horsman J A, Barquera B, Rumbley J, Ma J, Gennis R B (19994) The superfamily of heme-copper respiratory oxidases. J Bacteriol 176:5587-5600

Iwata S, Ostermeier C, Ludwig B, Michel H (1995) Structure at 2.8 Á resolution of cytochrome c oxidase from Paracoccus denitrificans. Nature 376:660-669

Iwata S, Saynovits M, Link T A, Michel H (1996) Structure of a water-soluble fragment of the “Rieske” iron-sulfur protein of the bovine heart mitochondrial cytochrome bc₁ complex determined by MAD phasing at 1.5 Á resolution. Structure 4:567-579

Iwata S, Lee J W, Okada K, Lee J K, Iwata M, Rasmussen B, Link T A, Ramaswamy S, Jap B K (1998) Complete structure of the 11-subunit bovine mitochondria cytochrome bc₁ complex. Science 281:64-71

Keilhauer C, Eggeling L, Sahm H (1993) Isoleucine synthesis in Cornebacterium glutamicum: molecular analysis of the ilvB-ilvN-ilvC operon. J Bacteriol 175:5595-5603

Kullik I, Giachino P (1997) The alternative sigma factor o^{B} in Staphylococcus aureus: regulation of the sigB operon in response to growth phase and heat shock. Arch Microbiol 167:151-159

Kusumotot K, Sakiyama M, Sakamoto J. Noguchi S, Sone N (2000) Menaquinol oxidase activity and the primary structure of cytochrome bd from the amino-acid fermenting bacterium Corynebacterium glutamicum. Arch Microbiol 173:390-397

Laemmli U K (1970) Cleavage of structural proteins during the assembly of the head of bacteriophage T4. Nature 227:680-685

Link A J, Phillips D, Church G M (1997) Methods for generating precise deletions and insertions in the genome of wild-type Escherichia coli: application to open reading frame characterization. J. Bacteriol-179:6228-6237

Matsushita K, Yamamoto T, Toyama H, Adachi O (1998) NADPH oxidase system as a superoxide-generating cyanide-resistant pathwax in the respiratory chain of Corynebacterium glutamicum. Biosc Biotechnol Biochem 62:1968-1977

Miller M J, Gennis R (1985) The cytochrome d complex is a coupling site in the aerobic respiratory chan of Escherichia coli. J Biol Chem 260:14003-14008

Molenaar D, van der Rest M, Petrovic S (1998) Biochemical and genetic characterization of the membrane-associated malate dehydrogenase (acceptor) from Corynebacterium glutamicum. Eur J Biochem 254:195-403

Molenaar D, van der Rest M E, Drysch A, Yücel R (2000) Functions of the membrande-associated and cytoplasmic malate dehydrogenases in the citric acid cycle of Corynebacterium glutamicium. J Bacterio 182:6884-6891

Nicholls D G, Ferguson S J (1992) Bioenegertics, 2^{nd} edn. Academic, London

Oehlmann W, Auling G (1999) Ribonucleotide reductase (RNR) of Corynebacterium glutamicum. ATCC 13032-genetic characterization of a second class IV enzyme. Microbiology 145:1595-604

Puustinen A, Finel M, Haltia T, Gennis R B, Wikstroöm M (1991) Properties of the two terminal oxidases of Escherichia coli. Biochemistry 30:3936-3942

Raitio M, Pispa J M, Metso T, Saraste M (1990) Are there isoenzymes of cytochrome c oxidase in Paracoccus denitrificans? FEBS Lett 261:431-435

Reinscheid D J, Kronemeyer W, Eggeling L, Eikmanns B J, Sahm H (1994) Stable expression of hom-1-thrB in Corynebacterium glutamicum and its effect on the carbon flux to threonine and related amino acids. Appl Environ Microbiol 60:126-132

Sahm H, Eggeling L, Eikmanns B, Krämer R (1995) Metabolic design in amino acid producing bacterium Corynebacterium glutamicum. FEMS Microbiol Rev 16:243-252

Sambrock J, Fritsch E F, Maniatis T (19*89) Molecular Cloning, A laboratory manual. Cold Spring Harbor Laboratory, Cold Spring Harbor, N.Y.

Sanger F, Nicklen S, Coulson A R (1977) DNA sequencing with chain-terminating inhibitors. Proc Natl Acad Sci USA 74: 5463-5467

Schäfer A, Tauch A, Jäger W, Kalinowski J, Thierbach, Pühler A (1994) Small mobilizable multi-purpose cloning vectors derived from the Escherichia coli plasmids pK18 and pk19: selection of defined deletions in the chromosome of Corynebaterium glutamicum. Gene 145:69-73

Schägger H, Pfeiffer K (2000) Supercomplexes in the respiratory chains of yeast and mammalian mitochondria. EMBO J 19:1777-1783

Schägger H, Borchart U, Machleidt W, Link T A, von Jagow G (1987) Isolation and amino acid sequence of the “Rieske” iron sulfur protein of beef heart ubiquinol: cytochrome c reductase. FEBS Lett 219:161-168

Schirawski J, Unden G (1998) Menaquinone-dependent succinate dehydrogenase of bacteria catalyzes reversed electron transport driven by the proton potential. Eur J Biochem 257:210-215

Smith P K, Krohn R I, Hermanson G T, Mallia A K, Gartner F H, Provenzano M D, Fujimoto E K, Goeke N M, Olson B J, Klenk D C (1985) Measurement of protein using bicinchoninic acid. Anal Biochem 150:76-85

Sone N, Sekimachi M, Kutho E (1987) Identivication and properties of a quinol oxidase supercomplex composed of a bc₁ complex and cytochrome oxidase in the thermophilic bacterium PS3. J Biol Chem 262:15386-15391

Southern E M (1975) Detection of specific sequences among DNA fragments separated by gel electrophoresis. J Mol Biol 98:503-517

Steinrücke P, Gerhus E, Ludwig B H (1991) Paracoccus denitrificans mutants deleted in the gene for subunit II of cytochrome c oxidase also lack subunit I. J Biol Chem 166:7676-7681

Tjp,as ÜE. Ryam D. Öevom W (1980) An improved staining procedure for the detection of the peroxidase activity of cytochrome P-450 on sodium dodecyl sulfate polyacrylamide gels. Anal Biochem 75:168-175

Trutko S M, Kuznetsova N N, Balitskaya R M, Akimenko V K (1982)Effect of the glutamic acid oversynthesis on the development of cyanide-resistant respiration in the bacterium Corynebacterium glutamicum. Biokhimiya 47:1608-1617

Van der Rest M E, Lange C, Molenaar D (1990) A heat shock following electroporation induces highly efficient transformation of Corynebacterium glutamicum with xenogeneic plasmid DNA. Appl Microbiol Biotechnol 52:541-545

Wakabayashi S, Matsubara H, Kim C H, King T E (1982) Structural studies of bovine heart cytochrome c₁ J Biol Chem 257:9335-9344

Xia D, Yu C, Kim H, Xia J, Kachurin A M, Zhang L, Yu L, Deisenhofer J (1997) Crystal structure of the cytochrome bc1 complex from bovine heart mitochondria. Science 277:60-66

Yiong J, Inoue K, Bauer C E (1998) Tracking molecular evolution of photosynthesis by characterization of a major photosynthesis gene cluster from Heliobacillus mibilis. Proc Natl Acad SciUSA 95:14851-14856

Yanisch-Perron C, Cieira J, Messing J (1985) Improved M13 phage cloning vectors and host strains: nucleotide sequence of the M13mp18 and pUC19 vectors. Gene 33:103-119

Zhang Z, Huang L, Shulmeister V M, Chi Y, Kim K K, Hung L, Crofts A R, Berry E A, Kim S (1998) Electron transfer by domain movement in cytochrome bcl. Nature 192:677-684

1. An isolated polynucleotide from coryneform bacteria containing a polynucleotide sequence coding for the ctaD gene, which is chosen from the group
a) a polynucleotide that is at least 70% identical with a polynucleotide that codes for a polypeptide that contains the amino acid sequence of SEQ ID No. 2,
b) a polynucleotide that codes for a polypeptide that contains an amino acid sequence that is at least 70% identical to the amino acid sequence of SEQ ID No. 2,
c) a polynucleotide that is complementary to the polynucleotides of a) or b), and
d) a polynucleotide containing at least 15 successive nucleotides of the polynucleotide sequence of a), b) or c),
where the polypeptide preferably has the activity of the cytochrome aa₃ oxidase subunit I.

2. An isolated polynucleotide from coryneform bacteria containing a polynucleotide sequence coding for the ctaE gene, which is chosen from the group
a) a polynucleotide that is at least 70% identical with a polynucleotide that codes for a polypeptide that contains the amino acid sequence of SEQ ID No. 4,
b) a polynucleotide that codes for a polypeptide that contains an amino acid sequence that is at least 70% identical to the amino acid sequence of SEQ ID No. 4,
c) a polynucleotide that is complementary to the polynucleotides of a) or b), and
d) a polynucleotide containing at least 15 successive nucleotides of the polynucleotide sequence of a), b) or c),
where the polypeptide preferably has the activity of the cytochrome aa₃ oxidase subunit III.

3. An isolated polynucleotide from coryneform bacteria containing a polynucleotide sequence coding for the qcrC gene, which is chosen from the group
a) a polynucleotide that is at least 70% identical with a polynucleotide that codes for a polypeptide that contains the amino acid sequence of SEQ ID No. 6,
b) a polynucleotide that codes for a polypeptide that contains an amino acid sequence that is at least 70% identical to the amino acid sequence of SEQ ID No. 6,
c) a polynucleotide that is complementary to the polynucleotides of a) or b), and
d) a polynucleotide containing at least 15 successive nucleotides of the polynucleotide sequence of a), b) or c),
where the polypeptide preferably has the activity of cytochrome c₁.

4. An isolated polynucleotide from coryneform bacteria containing a polynucleotide sequence coding for the qcrA gene, which is chosen from the group
a) a polynucleotide that is at least 70% identical with a polynucleotide that codes for a polypeptide that contains the amino acid sequence of SEQ ID No. 8,
b) a polynucleotide that codes for a polypeptide that contains an amino acid sequence that is at least 70% identical to the amino acid sequence of SEQ ID No. 8,
c) a polynucleotide that is complementary to the polynucleotides of a) or b), and
d) a polynucleotide containing at least 15 successive nucleotides of the polynucleotide sequence of a), b) or c),
where the polypeptide preferably has the activity of the Rieske Fe—S protein.

5. An isolated polynucleotide from coryneform bacteria containing a polynucleotide sequence coding for the qcrB gene, which is chosen from the group
a) a polynucleotide that is at least 70% identical with a polynucleotide that codes for a polypeptide that contains the amino acid sequence of SEQ ID No. 10,
b) a polynucleotide that codes for a polypeptide that contains an amino acid sequence that is at least 70% identical to the amino acid sequence of SEQ ID No. 10,
c) a polynucleotide that is complementary to the polynucleotides of a) or b), and
d) a polynucleotide containing at least 15 successive nucleotides of the polynucleotide sequence of a), b) or c),
where the polypeptide preferably has the activity of cytochrome b.

6. An isolated polynucleotide from coryneform bacteria containing a polynucleotide sequence coding for the nuoU gene, which is chosen from the group
a) a polynucleotide that is at least 70% identical with a polynucleotide that codes for a polypeptide that contains the amino acid sequence of SEQ ID No. 12,
b) a polynucleotide that codes for a polypeptide that contains an amino acid sequence that is at least 70% identical to the amino acid sequence of SEQ ID No. 12,
c) a polynucleotide that is complementary to the polynucleotides of a) or b), and
d) a polynucleotide containing at least 15 successive nucleotides of the polynucleotide sequence of a), b) or c),
where the polypeptide preferably has the activity of the NADH dehydrogenase subunit U.

7. An isolated polynucleotide from coryneform bacteria containing a polynucleotide sequence coding for the NuoV gene, which is chosen from the group
a) a polynucleotide that is at least 70% identical with a polynucleotide that codes for a polypeptide that contains the amino acid sequence of SEQ ID No. 14,
b) a polynucleotide that codes for a polypeptide that contains an amino acid sequence that is at least 70% identical to the amino acid sequence of SEQ ID No. 14,
c) a polynucleotide that is complementary to the polynucleotides of a) or b), and
d) a polynucleotide containing at least 15 successive nucleotides of the polynucleotide sequence of a), b) or c),
where the polypeptide preferably has the activity of the NADH dehydrogenase subunit V.

8. An isolated polynucleotide from coryneform bacteria containing a polynucleotide sequence coding for the NuoW gene, which is chosen from the group
a) a polynucleotide that is at least 70% identical with a polynucleotide that codes for a polypeptide that contains the amino acid sequence of SEQ ID No. 16,
b) a polynucleotide that codes for a polypeptide that contains an amino acid sequence that is at least 70% identical to the amino acid sequence of SEQ ID No. 16,
c) a polynucleotide that is complementary to the polynucleotides of a) or b), and
d) a polynucleotide containing at least 15 successive nucleotides of the polynucleotide sequence of a), b) or c),
where the polypeptide preferably has the activity of the NADH dehydrogenase subunit W.

9. A method for producing metabolic products, which is characterized by the fact that the following steps are carried out:
a) fermentation of the bacteria producing the desired metabolic product, in which one or more of the genes chosen from the group ctaD, ctaE, qcrC, qcrA, qcrB, nuoU, nuoV and nuoW is weakened or enhanced.
b) enrichment of the desired metabolic products in a medium or in the cells of the bacteria, and
c) isolation of the metabolic products.

10. A method as in claim 9, which is characterized by the fact that the metabolic product is an amino acid chosen from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan, L-arginine.

11. A method as in claim 9, which is characterized by the fact that the metabolic product is an organic acid, preferably chosen from the group acetic acid, citric acid, isocitric acid, lactic acid, succinic acid, fumaric acid, ketoglutaric acid, pyrotartaric acid and malic acid.

12. A method as in claim 9, which is characterized by the fact that the metabolic product is a vitamin.

13. A method as in claim 9, which is characterized by the fact that the metabolic product is a nucleoside or nucleotide.

14. A method as in claim 9, which is characterized by the fact that the metabolic product is a mono- or polyhydric alcohol.

15. A method for detecting RNA, cDNA and DNA in order to isolate nucleic acids, or polynucleotides or genes, which is characterized by the fact that the polynucleotide sequences in accordance with claims 1 to 8 are used as hybridization probes.

16. A method as in claim 15, which is characterized by the fact that the hybridization is carried out under stringency corresponding to a maximum of 2×SSC.
